SEQUENCE LISTING (3 sequences)

SEQ ID NO: 1 - DNA, 5888 bp, Sus scrofa
  misc_feature (1)..(5888): Sequence for AMPK gamma subunit
atgagcttcctagagcaaggagagagccgttcatggccatcccgagctgtaaccaccagctcagaaagaagccatggggaccaggggaacaaggcctctagatggacaaggcaggaggatgtagaggaaggggggcctccgggcccgagggaaggtgagttcaaggccagttctggggagctgggactgggggcagtgggcagtcctcaaacctggggcccgtctctggtctggtccctccataacacaggcacataacatcatgcagccagtctccccacaagggggaggacaactgcattgctgatccaggggtccagggatccaaggtggccaactcaggacagagccactgtcttctctgtgactctctgagactcagctctctcacctgcaaaatggggccacagcattcaggcttcccaaggttgcaatgaggatgaatggagacagcagatgaggaagttctctggaagagggagttactgtgctctccctcccgctccccgaacaggtccccagtccaggccagttgctgagtccaccgggcaggaggccacattccccaaggccacacccttggcccaagccgctcccttggccgaggtggacaaccccccaacagagcgggacatcctcccctctgactgtgcagcctcagcctccgactccaacacagaccatctggatctgggcatagagttctcagcctcggcggcgtcgggggatgagcttgggctggtggaagagaagccagccccgtgcccatccccagaggtgctgttacccaggctgggctgggatgatgagctgcagaagccgggggcccaggtctacatgcacttcatgcaggagcacacctgctacgatgccatggcgaccagctccaaactggtcatcttcgacaccatgctggaggtgaggccacgcctcagccccccccatcctcacccccccccaggatgccttgccagctctgcccccctccaagccccttcccgaactccttccggcatgaatggagaccgggggagggcttctgctctctgcacgcaccccttaattgtcatcccagctctgcaactcagtatccagagataggaatgcctgctttagcctgcgaatttcagaggattcctgggacaagccaggcaatatatgaaagtctttgcagggtggcttaggacaaagagcaagggactcttggtaagagaaaaataggatgagctctgctccccactcttcccttaggttaaactatgaaacatttggttccgtgcttctcgctgtgtgcactatttgattctagtggaatatgaacaaatacatttcatgtagtagctttgtatgttataatattagatattttacaatattagaaaattacagtcagcaggtgtagatagtcttgtttagggtgaggcccaaataagtcaatgtaaaatttatttagggaaaaatattttgtaaatattatacacataatttcacctctagcacttaacaaaatcgatactatgtgtgtctgtacacttatgactttggagtagaaacactgggttggtttcccacaccttggagtgcttggggaggggtcacctcagtacctctggccaccagcagccttagatctggaacaaatgtgcagacaaggatctcgtggagggcatgccaggacgtgggagaggcagacagcaggctcatgtagaggcaggcccgggaggcgcccggtggaagaaccctggctggcaggggacctctgaggcgcagggaacgattcaccctcaactgttctctccggcgctcagatcaagaaggccttctttgccctggtggccaacggcgtccgagcggcacctttgtgggacagcaagaagcagagcttcgtgggtgaggaggggctggggaggcagaggtggtggggaagggaatagggggaccttgtggggtgattctagggccgagctctgacacaccacaggcttcaaccaagcaggggcctggcctggagagggggggagcatttgaccccggtctcctggtggccagctgggagatctcaactgtaggagagctgtgaccagctgacccctccagctctactaccccaaggtccctgtcgcaggtgctaagtaagaagaggacaggcggaggaaggaagtcagaaaatagaagaagcagggcaggaaggagagaaatgacaggggaagcataagagggacaaccccatttgtcaggcacgggaggggctgccctcctgtcctcttttggccaccctcagtaaaaggatgtgggcagggtggggggaggggcccgggctgacccccattgctcccctcgccccacagggatgctgaccatcacagacttcatcttggtgctgcaccgctattacaggtcccccctggtgaggagtggtctgggggtcctggaacacccatctgggctggggtggaaggagttcaggggaccctcgcctgactttgggagttccgttgctgtctttaggtccagatctacgagattgaagaacataagattgagacctggaggggtgagcaggcgaggggacgggcgaaggggctgagggtgtgtgggtgaggatggggccaaggacctcagggagagcatgcgcagtggaggtttcctggaggaagcgggaggagggtgatcgggagcccaggggatctaagggagggagacagtctgggggtggccacgtgaggcggggtggtcggcccctttgtgctgattctggcttttcctgcagagatctaccttcaaggctgcttcaagcctctggtctccatctctcccaatgacaggtgagcttccccagccgcccactcgagcctccttgccccgcacagaccccttctccagctcatcggttctaagctcatggactcatcgtccgtggactgcagatgcggcagctttgacaccctgtcctcctctccaggggggctgggatgaaggggctctctttccagactgccccaggctcactgctcccacctccacagcctgttcgaagctgtctacgccctcatcaagaaccggatccaccgcctgccggtcctggaccctgtctccggggctgtgctccacatcctcacacataagcggcttctcaagttcctgcacatctttgtgagcctgggcacagcctcagggacaacctgagtggctgagaagtcttcagccctagggatgggggagggagtagctgggagccccctgagggctactccctcctggcctcacctgtcccaacccaaccagggcaccctgctgccccggccctccttcctctaccgcaccatccaagatttgggcatcggcacattccgagacttggccgtggtgctggaaacggcgcccatcctgaccgcactggacatcttcgtggaccggcgtgtgtctgcgctgcctgtggtcaacgaaactggtacctatgcccaggatgggggctctggctgtgatgggactgcgggggggcaggggtctaggtggcatcaacttggggtccagcatggagtcagggctagcagtctctgccttctttgagctttggaccagttgcttagcctctctgagccagacctcaagttcttcctctgaaaaagacttaaaggaaccatggctgcacactgtttcaaggttaaattcaccataaagaagccagatatcgagaagtattttaatttatgtttgattatgaaacatttccaatgtctgaacatggcagaaaaaactataatgaaccccacgtatccacctggattaaccactgttaacatgatgccgtgaccagttcttttttttttttcgtggccaaagtaatttaaaggaaattatattatagaattatgtcatttcaccccgggacacttcatctgcctctcttaaaataagggtacttcctatatcaccttacaattatgaataatttattaatgctatctaatatccaatcctaattctcatttctccattttccccaagaatatcttttttttttttaacagttgatttgttgagaccaagatccaatcaaggtccatgtttcgcatttgcttcttttttcccttaagcctcttttaatctagaacagttccctccttgctttattttcgtgacaccggtgatgagaagctgggtcagttgtcctgtagaatgtcacactttgagagatttgcctgttagctttccacaggtagcccttattttttttctctattcctgcgtttcctgtgaccgggaaattagctctaaaggctggatcagattcaggctagacatttgaacctagaatatttcagaggtgatgccatgtactcctgtctcatcatattaggaggcatgacggcaggtgtgtctctctgtgtgatgctatttgatctgtgggctcaggtgctggccgtctgatgcctcactataaagccggtagcgtgaggggtggggagttcattcccaaaccccaccccaggccctcgctcannatcctggntctgacccaaacctctcccctgtctttctcacaccttcctccctgcccctcccatcccccacaggacaggtagtgggcctctactctcgctttgatgtgatcgtaagtatctatggggaacggaggggacctgggggaccacagggaggctgtggtgtgaagatggatggaggttggtatctgtggaccagggaggcctttacagtgtatatagagagattatttgtgggactggagcctggccgagggctaagaatggtcccccctccctgcccagcacctggctgcccaacaaacatacaaccacctggacatgaatgtgggagaagccctgaggcagcggacactgtgtctggaaggcgtcctttcctgccagccccacgagaccttgggggaagtcattgaccggattgtccgggaacaggtaccccagccccttcatgcctgctcccaacatgtagggccccgtcctcctcgtgagcagctccagctagcccatccaccgggcacctgtccggcccccccatcccccattctcatggccaagctcatggtgtccatattggccagtgactggtcctattatcggggccctcagggcaagggccacagccagctgatcacccagggtggtcacagccacccgtaagcagtttctaggagaccctctgaggcacccccagttaggttaagttgttgcccctgattctcagtgccaacctcattggccgccatagccgcatggcactgccccctcactgagcctctgtgggcccacaggtgcaccgcctggtgctcgtggatgagacccagcaccttctgggcgtggtgtccctctctgacatccttcaggctctggtgctcagccctgctggaattgatgccctcggggcctgagaaccttggaacctttgctctcaggccacctggcacacctggaagccagtgaagggagccgtggactcagctctcacttcccctcagccccacttgctggtctggctcttgttcaggtaggctccgcccggggcccctggcctcagcatcagcccctcagtctccctgggcacccagatctcagactggggcaccctgaagatgggagtggcccagcttatagctgagcagccttgtgaaatctaccagcatcaagactcactgtgggaccactgctttgtcccattctcagctgaaatgatggagggcctcataagaggggtggacagggcctggagtagaggccagatcagtgacgtgccttcaggacctccggggagttagagctgccctctctcagttcagttcccccctgctgagaatgtccctggaaggaagccagttaataaaccttggttggatggaatttccacactcg

SEQ ID NO: 2 - DNA, 421 bp, Sus scrofa
  misc_feature (1)..(421): Partial sequence for Porcine Leptin Receptor gene
gcactgtttgagcacttggaaagttaaataattattgttggagactgcatgttttaatcttagatacttcctatttatgtcttagtcaaaatgattaattgcttttctatgtgtcttttaaatgtcctaacagaatttatttatgtgataactgcatttgacttggcatatccaattactccttggaaatttaagttgtcttgcatgccaccaaatacaacatatgacttcctcttgcctgctggaatctcaaagaacacttcaactttgaatggacatgatgaggcagttgttgaaayggaacttaatwyaagtggtacctacttatcaaacttatcttctaaaacaactttccactgttgcttttggagtgaggaagataaaaactgctctgtacatgcagacaacattgcagggaagg

SEQ ID NO: 3 - protein, 96 aa, Sus scrofa
  MISC_FEATURE (56)..(56): Xaa = Met or Thr
  MISC_FEATURE (60)..(60): Xaa = Ser or Ile
EFIYVITAFDLAYPITPWKFKLSCMPPNTTYDFLLPAGISKNTSTLNGHDEAVVEXELNXSGTYLSNLSSKTTFHCCFWSEEDKNCSVHADNIAGK

(19) United States
(12) Patent Application Publication
(10) Pub. No.: US 2007/0105107 A1
(43) Pub. Date: May 10, 2007

(54) Marker assisted best linear unbiased prediction (ma-blup): software adaptions for large breeding populations in farm animal species

(75) Inventors: Tianlin Wang, Apex, NC (US); Michael M. Lohuis, Des Peres, MO (US); Cheryl J. Kojima, Knoxville, TN (US); Fengxing Du, St. Charles, MO (US); John C. Byatt, Ballwin, MO (US)

Correspondence Address: HOWREY LLP, C/O IP DOCKETING DEPARTMENT, 2941 FAIRVIEW PARK DRIVE SUITE 200, FALLS CHURCH, VA, 22042, US

(73) Assignee: MONSANTO TECHNOLOGY LLC, St. Louis, MO (US)

(21) Appl. No.: 10/561,075

(22) PCT Filed: Jan. 27, 2005
(86) PCT No.: PCT/US05/02362

§ 371 Date: Dec. 19, 2005

Related U.S. Application Data
(60) Provisional application No. 60/543,034, filed on Feb. 9, 2004.

(51) Int. Cl. C12Q 1/68; G06F 19/00

(52) U.S. Cl. 435/6; 702/20

(57) ABSTRACT

The invention provides methodologies for improved molecular genetic analysis of individual animals and animal populations. The invention includes methods and systems for identifying those animals in a population that are most likely to heritably pass on desirable traits. Provided are means for evaluating the estimated breeding values and increasing the average genetic merit for animals in a population. For each trait, the instant invention provides methods for evaluating the relative effect of one or more quantitative trait loci (QTL) and three or more molecular genetic markers for each QTL The relationship between these various markers and the pre-selected trait and QTL is calculated, along with the contribution of other factors such as pedigree and known measures with respect to quantitative trait, and these data are used to calculate estimated breeding values for the animals in the herd and to rank the animals according to these estimated breeding values.

This application claims the benefit of U.S. provisional application Ser. No. 60/543,034, filed Feb. 9, 2004, which is herein incorporated by reference.

### BACKGROUND OF THE INVENTION

1. Field of the Invention

The present invention relates generally to the field of improving genetic merit in animal species at both the individual animal and herd levels. Among the various embodiments, it particularly concerns a method for improving the genetics in swine and cattle herds. More particularly, the invention provides for the analysis of multiple genetic markers as part of a breeding and herd management program.

2. Description of Related Art

Owing to the rapidly growing and improving field of genomics, there is a need for a means of using newly available genotypic information to improve the development of commercial animal and plant products. Such a means must allow for the rapid genetic improvement of a population so as to optimize the short-term occurrence of desirable traits in the population without jeopardizing the potential for long-term genetic improvement (e.g. as has been documented by excessive inbreeding or intense selection pressure on a limited number of genes or quantitative trait loci (QTL) [e.g. Gibson, 1994]). Such a method would need to provide a means for quickly and efficiently maximizing the usefulness of new understanding regarding the function of various genes and/or combination of genes; while at thee same time optimizing the use of phenotypic, genotypic (e.g. SNPs) and pedigree information This is particularly important in traits where the phenotypes are difficult or expensive to measure (e.g. feed intake or disease resistance/tolerance), traits that are measured late in life or at the end of life (e.g. longevity or meat quality) or measurable only in one sex (e.g. milk yield, litter size or maternal or paternal calving ease). In traits such as meat quality, not only is the trait measured after selection decisions have already been made, but the animal has most likely been slaughtered to enable trait measurement and, therefore, is no longer available for selection. In these cases, Marker-Assisted Selection (MAS) can provide extremely useful information for selection prior to the availability of phenotypic measures. The present invention provides the ability to practice MAS on several QTL in an optimal and efficient manner at an industry scale.

### SUMMARY OF THE INVENTION

The instantly disclosed invention solves previously existing problems by providing a method that allows for the input of pedigree, phenotypic, and molecular genetic metrics for a breeding population, provides for the concurrent and interdependent evaluation of these factors, for each animal (or plant), and then provides a ranking of the individuals in the population that enables optimal weighting of all sources of information to achieve the desired breeding goals.

The instantly disclosed invention solves the deficiencies associated with previously available methodology by allowing for the concurrent evaluation of one or more, two or more, or three or more molecular genetic markers, pedigree information, and, optionally quantitative trait metrics through the use of iteration-on-data (IOD) algorithms that dramatically reduce computer memory requirements and preconditioned conjugate gradient (PCCG) algorithms, with variable-size diagonal blocking as a preconditioner, that dramatically reduce computing time. The invention also provides algorithms to compute inbreeding coefficients at QTL. Existing software that may have the capability to incorporate marker information is severely hampered by long computing times and excessive computer memory requirements. By dramatically reducing the computer memory requirements to solve mixed-model equations via the incorporation of IOD algorithms, various aspects of the instant invention makes it possible to include a virtually unlimited number of marked QTL and any number of traits. The PCCG algorithms included in aspects of the instant invention significantly reduce computing time, thereby allowing larger numbers of markers and traits to be included in the mixed model equations while reaching adequately converged solutions in a time period acceptable to breeding programs operating at an industry-scale. The significance of being able to practically and efficiently include more markers has two main advantages. First, as more marked QTL are included in MA-BLUP (marker-assisted best linear unbiased prediction) a greater proportion of the genetic variance of selected traits can be explained by the marker information and, therefore, genetic progress is further accelerated. Secondly, it has been shown that intense selection at only a few QTL (e.g. 1 to 3 loci) can accelerate short-term genetic response, but this occurs at the expense of long-term genetic progress. In fact, it has been shown that MAS (marker assisted selection) with only a few loci included can provide less favorable long-term genetic response than BLUP alone (i.e. no marker information included) (Gibson, 1994). Therefore, if selection can take place at several markers simultaneously, as is provided by the instant invention, the loss of long-term response is minimized.

In various aspects of the invention the trait(s) sought to be improved are selected for the presence of desirable characteristics, including but not limited to: the presence or absence of specific gene or marker variants or alleles, health traits, reproduction traits, meat quality traits, efficient growth traits, or any other desired phenotypic trait.

Various embodiments of the instant invention provide for a method of increasing an animal population's genetic merit with respect to one or more pre-selected traits. Certain aspects of this method comprise the steps selecting one, two, three, or more molecular genetic markers of interest, for each of one or more quantitative trait loci (QTL), for each trait for which improvement is desired. For each of the selected characteristics, whether as molecular genetic marker genotypes or quantitative trait measures, a computer readable database is provided that indicates each the status of the animals in the population with respect to the selected characteristic if available for the animal. The methods and systems of the present invention do not require phenotypes to be available for every animal in the population (that is the methods and systems of the present invention are capable of handling missing terms). In addition, due to its multiple-trait capabilities, of the present invention does not require phenotypes to be available for all traits for a given animal to be effective. It is of particular note, that the invention does not require genotypes for every animal or for every marker to be effective. For example, even if genotypes are available only on the most recent generations in the pedigree and available for some markers or animals but not for others, the methods and systems of the instant invention can still be remarkably effective.

Additionally, a computer readable database providing the pedigree for each animal in the population may also be provided. A computer is then used to perform a molecular genetic marker-assisted best linear unbiased prediction (MA-BLUP) analysis of the data in the databases provided. This analysis simultaneously produces estimates of breeding value (EBV) for each animal and for each trait using marker, pedigree, and phenotypic data, if available, on all traits simultaneously. A ranking of the animals in the population is then produced wherein the animals are ranked according to their respective EBV (estimated breeding value) for the combination of the individual trait EBVs that are represented in the selection index for any given population, which take into account inbreeding coefficients for the selected traits. This ranking may then be used as part of an animal management or breeding plan to optimize the improvement of the population's average genetic merit for the selected characteristics.

Other embodiments of the invention provide for a system for increasing an animal populations average genetic merit. In various aspects of this embodiment the system comprises a computer, one or more computer accessible databases, a computer executable program, and a user interface. The databases, computer, and computer program provided by the various aspects of this embodiment of the invention are the same as those in the methods described supra. User interfaces considered to be useful for the various aspects of this embodiment of the invention are configured so as to be coupled with the computer so as to allow the user to instruct the computer to access the available databases and allow the computer program to used the computer's processor to generate, as output their individual estimated breeding value and/or one or more rankings of the animals in the population.

Another embodiment of the instant invention provides for a method of evaluating an animal population's breeding value or genetic merit for a pre-selected set of characteristics. Although the evaluation may be accomplished using one or two molecular genetic markers for each QTL, according to various preferred aspects of this invention the characteristics will typically include at least three molecular genetic markers. Even more preferably, the selected characteristics will include four or more molecular genetic markers. The selected characteristics will be linked (or associated) with one or more QTLs or one or more genes of economic value. Various aspects of this embodiment of the invention provide for the steps of: (a) selecting one, two, three, or more molecular genetic markers of interest that are linked to one or more QTLs or genes; (b) providing databases comprising data for individual animals in the population, that include the animals pedigree, and the animal's status for each of the selected trait, where known; (c) using a computer executable program on a computer capable of performing MA-BLUP to simultaneously analyze the data from the databases provided to produce a ranking of each animal, in the population, according to its EBV for the selected traits, taking into account possible inbreeding; and finally (d) evaluating the individual trait EBV's to determine the combined multi-trait EBV for the selected traits in the selection index.

Thus, as provided herein, the MA-BLUP executes a “joint” or simultaneous analysis to produce EBVs for each trait and each animal from the mixed model equations. These are then used in combination by MA-BLUP to provide a single value known as the “Selection Index.”

Other embodiments of the instant invention provide for systems useful for increasing an animal population's genetic merit, where the system comprises the following components. (a) A computer to which data is input and which is capable of running a computer program to produce output data. (b) At least one computer accessible databases, where the databases are selected from those providing pedigree data for the population, databases providing information on quantitative trait loci and molecular genetic markers (both those markers known to be associated with any selected quantitative trait loci. (c) A computer executable program capable of simultaneously evaluating the data in all databases provided and producing as program output estimated breeding values (EBVs) for each trait and for each individual animal in the population for each trait individually and in combination and of ranking the animals according to their respective EBVs. (d) A user interface including data input and retrieval systems, where the user interface is coupled to the computer and configured to allow the user to instruct the computer to access any combination of the available databases and use the computer program to generate the output rankings and individual animal estimated breeding values.

Other embodiments provide for using any of the methods or systems described herein to evaluate the average genetic merit of an animal population for one or more selected traits.

Yet another embodiment of the instant invention provides a method for identifying the best breeding pairs in a defined animal population to allow for optimal improvement of a pre-selected trait in the population (e.g. to quickly improve the average EBV for that characteristic in the population). According to this aspect of the invention, any of the methods for estimating animal or herd EBVs for a given trait may be used as part of a method to identify those pairs of animals best suited for crossing (without exceeding an acceptable rate or degree of inbreeding) so as to optimize the increase of the population's average breeding value or genetic merit for a pre-selected characteristic or trait.

Taken together, the MA-BLUP methods and systems of the instant invention provide for a synergistic confluence of elements that enable those skilled in the art to solve the mixed model equations that were previously intractable (or impractical to solve for industry-scale populations) problem of manipulating pedigree, QTL, and molecular genetic marker data to calculate the EBV for each animal in a vary large population of more than one million animals and rank each animal in that population according to their individual EBV for one or more pre-selected traits.

Other embodiments of the instant invention provide methods for enhancing one or more meat quality traits, wherein the meat quality traits include, but are not limited to loin and/or ham pH, color, tenderness, marbling and water-holding capacity. Various aspects of these embodiments provide methods for screening a plurality of pigs to identify the status of each animal with respect to one or more single nucleotide polymorphisms (SNPs) in the porcine PRKAG3 gene (the PRKAG3 gene encodes a muscle-specific isoform of the regulatory gamma subunit of adenosine monophosphate-activated protein kinase (AMPK), PRKAG3 stands for protein kinase AMP-activated gamma-3 subunit). Preferably the SNPs identified are selected from the group consisting of: an A/G at position 51, A/G at position 462, A/G at position 1011, C/T at position 1053, C/T at position 2475, A/G at position 2607, A/G at position 2906, A/G at position 2994, and C/T at position 4506, wherein all numbering is according to the sequence of SEQ ID NO:1. Once those animals having at least one desired allele are identified, they are selected for use as sires/dams in a breeding plan designed to produce offspring having an increase frequency of the desired allele.

Other embodiments provide for methods and/or kits for detecting the PRKAG3 SNPs described above. Furthermore, in various aspects of these embodiments these methods and/or kits are used as components of a general method or system that incorporates the use of the MA-BLUP analysis described herein. Use of the MA-BLUP integrating methods and systems provides breeding herd managers the means necessary to create a herd management and breeding plan to more rapidly improve the meat quality traits effected by the porcine PRKAG3 gene. Particular aspects of this embodiment provide for methods of screening a population of animals to identify those animals that when mated together are likely to produce offspring exhibiting improvement in at least one desirable meat quality trait. In a particularly preferred aspect of this embodiment the desired meat quality trait is selected for higher ham or loin pH, darker color, greater tenderness, more marbling and/or increased water-holding capacity, or any combination thereof.

As noted various embodiments of the instant invention provide for kits useful for carrying out the instant invention. Various aspects of these embodiments specifically provide for kits that are useful for the detection of SNPs in the porcine PRKAG3 gene.

### BRIEF DESCRIPTION OF THE DRAWINGS

The described drawings form part of the present specification and are included to further demonstrate certain aspects of the present invention. The invention may be better understood by reference to one or more of these drawings in combination with the detailed description of specific embodiments presented herein.

FIG. 1: FIG. 1 provides a schematic representation of the inputs and output of the MA-BLUP program (MA-BLUP is represented as a “black box”).

FIG. 2: FIG. 2 provides a flow diagram of representing one possible algorithm for implementing the MA-BLUP program described herein.

FIG. 3: FIG. 3 provides a flow chart representing one possible algorithm for solving the mixed model equations (MME). This is expanded version of the step enclosed in the rhomboid in FIG. 2.

FIG. 4: The DNA sequence of the Sus scrofa AMPK gamma subunit (PRKAG3) (SEQ ID NO:1), as provided available as Genbank accession number AF214521.

FIG. 5: A graph depicting genotype values for SNP assays 1484004 and 148009.

FIG. 6: A graph depicting breeding values for SNP assays 1484004 and 148009.

FIG. 7: DNA and amino acid sequence of portion of Sus scrofa leptin receptor (pLEPR) gene that contains the M69T and S73I polymorphisms. The single nucleotide polymorphisms and accompanying amino acid changes are shown in bold. Nucleotide sequence without accompanying amino acid sequence is intronic. The sequence starts at position 311 of Genbank accession AF184172, “Sus scrofa leptin receptor (LEPR) gene, exon 4 and partial coding sequence”. The M69T polymorphism is at nucleotide position 609 of sequence at Genbank accession AF184172.

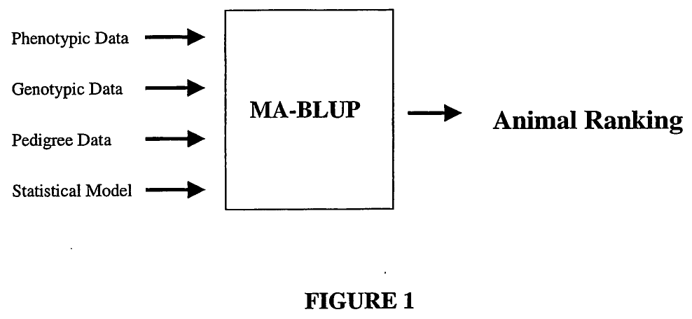

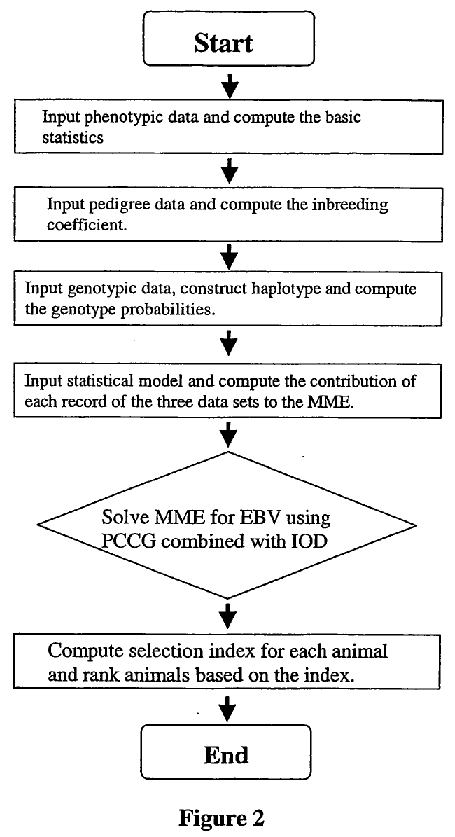

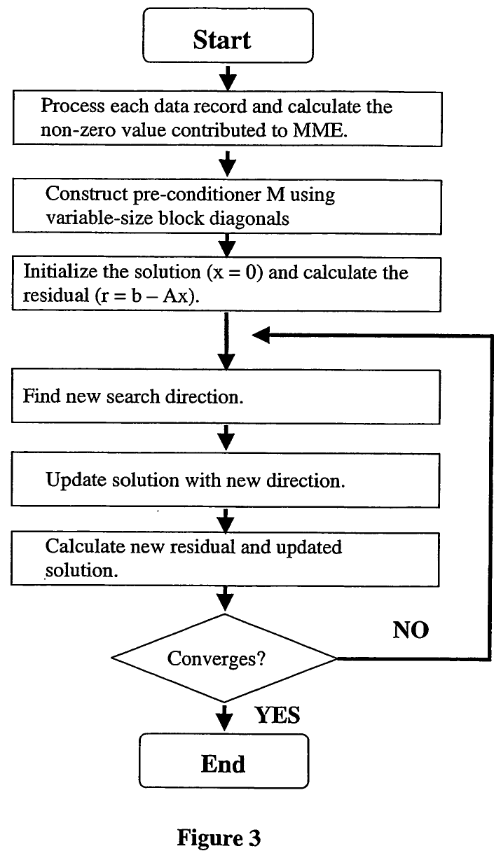

### DESCRIPTION OF ILLUSTRATIVE EMBODIMENTS

The instantly disclosed invention sets forth a method for the rapid improvement of an animal or plant population, based on pedigree, phenotypic and/or genotypic information. Thus, using the instantly disclosed invention, one of ordinary skill in the art will be able to use newly described genetic or phenotypic information in order to produce offspring optimized for one or more desired traits and/or to increase the population's genetic merit for a desired and/or pre-selected characteristic or trait. This phenotypic/genotypic information may be obtained from a variety of sources. Such sources include, but are not limited to marker genotypes on some or all of the animals in the breeding population, new or accumulated pedigree information and/or phenotypic trait measurement data and new biometric techniques.

The instant invention also provides for methods, compositions, and kits useful for improving the meat quality traits in a swine population. Specifically, the instant invention provides for methods, compositions, and kits useful for the analysis of an animals status with respect to the porcine PRKAG3 gene. Nevertheless, one of ordinary skill in the art will appreciate that the systems and methods described herein (including the MA-BLUP methodology) can be effectively used with all known quantitative trait loci and all known molecular genetic markers. By way of example, the invention provided herein can make effective use of polymorphisms in the melanocortin-4-receptor (MC4R) gene and the PRKAG3 gene.

For the sake of simplicity the language and examples used in the present disclosure will primarily refer to animal populations. Nevertheless, in view of the present disclosure, those of skill in the art will appreciate that the claimed inventions could be modified for use in plants by those skilled in the art who have access to the present disclosure.

Defied Terms

The following definitions are provided herein in order to aid the quantitative or molecular geneticist or animal breeder of ordinary skill in more easily and fully appreciating the instant invention. As suggested in the definitions provided below, the definitions provided are not intended to be exclusive, unless so indicated. Rather, they are provided as preferred definitions, provided to focus the skilled artisan on various illustrative embodiments of the invention.

As used herein the term “acceptable rate of inbreeding” preferably means a level of inbreeding where the benefits of inbreeding outweigh any negative effects. In general, inbreeding will accumulate in an animal population as a result of intra-population selection. Typically, there is an inverse relationship between rate of inbreeding (ΔF) and rate of genetic progress (ΔG). The optimum ΔF is the rate at which inbreeding is allowed to accumulate in order to optimize both short-term and long-term genetic gains. Under standard practice in swine it is typically desired that AF be held to less than 1% per year. Methods to approximate AF are given, infra, in the “Illustrative Embodiments” section.

As used herein the term “allele” refers to a particular version or variant of a specified gene.

As used herein the term “BLUP” (which is an acronym for best linear unbiased prediction) refers to a statistical methodology introduced by Henderson (1959, 1963) that has become an animal breeding industry standard for predicting breeding values for individual animals.

With standard post-graduate training in animal breeding techniques, BLUP can be performed, by those of ordinary skill in the art, using any of the various commercially available computer programs that are used for genetic evaluation of an animal and/or herd. Most currently available programs are customized programs designed specifically to meet the needs of the breeding company. However, some standard software packages that are publicly available can be used to perform BLUP (e.g. “MTDF-REML” from Curt Van Tassell (curtvt@aipl.arsusda.gov); “PEST” from Eildert Groeneveld (eg@tzv.fal.de); “DMU” from Just Jensen (lofjust@vm.uni-c.dk); “MATVEC” from Steve Kachman (www.statistics.unl.edu/faculty/steve/software/matvec/); and “BLUPF90” from Ignacy Misztal (http://nce.ads.uga.edu/˜ignacy/newprograms.html)). Typical input parameters for BLUP programs include genetic and phenotypic parameter estimates, phenotypes, pedigrees, and fixed effects. BLUP models can be described most easily in matrix notation as follows:
y=Xβ+Za+e,
where, y is the vector of phenotypic observations; β is a vector of fixed effects; X is an incidence matrix relating β to y; a is a vector of animal effects with a mean of zero and a variance-covariance matrix Gₐ; Z is an incidence matrix relating a to y; and e is a vector of residual effects with variance-covariance matrix R. Gₐ can be modeled as Gₐ=A δ²ₐ, where A is the additive relationship coefficient matrix between animals, and δ²ₐ is the additive genetic variance. One of the requirements to obtain BLUP is to obtain the inverse of Gₐ, which can be computed very efficiently even with extremely large data sets (Henderson, 1976; Quaas et. al., 1984; Quaas, 1988).

As used herein the term “breeding plan” preferably refers to a program for improving herd genetics using the information provided by the methods and systems described herein.

As used herein the term “breeding value” preferably refers to the expected value of an animal as a parent. It is also a measure of the animal's net breeding value. Half of the breeding value is transmitted to its progeny, and this portion can be referred to the expected progeny difference (EPD) or estimated transmitting ability (ETA). These measures of breeding value are typically expressed as a difference of the present population mean or the population mean at a fixed point in time (see, Van Vleck, p. 186).

As used herein the term “closeness,” when used to describe a molecular genetic marker and QTL, preferably refers to the relative linkage distance or probability of recombination between the marker locus and the locus responsible for the trait in a unit of Morgan (M).

As used herein the term “drip loss” preferably refers to the change in weight of a cut of meat (e.g. loin chop) due to loss of moisture to absorbent packaging materials over a specified time period, especially while the meat sits in a display case.

As used herein the term “economic trait locus” (ETL) preferably refers to a location on a chromosome that is linked to a “quantitative trait” providing economic value.

As used herein the terms “efficient growth traits” and/or “performance traits” preferably refers to a group of traits that are related to growth rate and/or body composition of the animal. Examples of such traits include, but are not limited to: average daily gain, average daily feed intake, feed efficiency, back fat thickness, loin muscle area, and lean percentage.

As used herein the term “estimated breeding value” (EBV) preferably refers to a specific numeric value for an animal that predicts its “breeding value”. EBV is often calculated using commercially available analysis programs (the output from BLUP and marker assisted BLUP (MA-BLUP) programs are examples of EBVs).

As used herein the term “gene” refers to a sequence of DNA responsible for encoding the instructions for making a specific protein within a cell or may also include instructions for when, where, and in what abundance a protein is expressed).

A used herein the term “genetic merit” refers to the value of the germplasm for providing a desired trait. That is, the greater the genetic merit of an animal for a given trait, the more likely it is to provide offspring having the desirable trait.

As used herein the term “fixed effects” preferably refers seasonal, spatial, geographic, environmental or managerial influences that cause a systematic effect on the phenotype or to those effects with levels that were deliberately arranged by the experimenter, or the effect of a gene or QTL allele/variant that is consistent across the population being evaluated.

As used herein the term “half-sib” refers to a group of animals all sharing one parent. Specifically, the term is most frequently used as “paternal half-sib”, which refers to offspring sharing the same sire.

As used herein the term “health traits” preferably includes any traits that improve the health of the animal and/or herd. These include, but are not limited to: the absence of undesirable physical abnormalities or defects (like scrotal ruptures in pigs), improvement of feet and leg soundness, resistance to specific diseases or disease organisms, or general resistance to pathogens.

As used herein the terms “herd” and “population” refer to any group of breeding animals having a sufficient number of animals for the effective use of the instant invention. The term may apply to animals such as swine, cattle, goats, or any other animal that is raised commercially, including, but not limited, to fowl (such as turkeys or chickens) or any other species where it is desirable, for any reason, to analyze multiple traits in creating a breeding program. Moreover, the term population may also be used to refer to a plant population.

As used herein the term “improved germplasm” preferably refers to change in the genome, improved frequency of genetic markers, genes, alleles of markers or genes, or any combinations of multiple markers or genes that is preferred over other forms of the genome that exist in the population. This includes forms of the genome that result in improved breeding values, but for which genotypes are not known. The term may, depending on the context, be used to refer to the genetic makeup of either a single animal or to the genetics of a herd, considered as a whole. Thus, the term “improved germplasm” covers both the introduction of a preferred trait in an individual and an increase in frequency of expression of a desired allele within a herd.

As used herein the term “inbreeding coefficient at a QTL” preferably refers to the probability of two alleles at a QTL being identical by descent. These inbreeding coefficients are used in the calculation of Gᵥ⁻¹ The algorithm used to compute the inbreeding coefficient for a QTL is base on the method described in Abel-Azim and Freeman (2001).

As used herein, the term “informativeness,” when used to describe or modify the term “molecular genetic marker” preferably refers to a measure of the marker's value as a predictive determinant for how likely a given trait and/or QTL is to be inherited by the animal's offspring. Thus, informativeness is a measure of the genotypic variation present at the marker locus and is determined as a measure of the heterozygosity frequency of the marker. If a marker is sufficiently informative and located relatively close to the QTL location, the usefulness as a marker for a QTL is increased. The more informative the markers are that surround a QTL, the more closely the QTL locus can be defined.

As used herein the term “locus” refers to a specific location on a chromosome (e.g. where a gene or marker is located). “Loci” is the plural of locus.

As used herein the term “MA-BLUP” (an acronym for marker-assisted BLUP) is a method of analysis that utilizes the same inputs as BLUP (see above) and additionally adds the animal's marker genotype to the calculus. As with BLUP, MA-BLUP models can be described most easily in matrix notation as follows:
y=Xβ+ZKυ+Zu+e
where, y is the vector of phenotypic observations; β is a vector of fixed effects; X is an incidence matrix relating β to y; υ is the vector of additive effects at the marked QTL with a mean of zero and a variance-covariance matrix Gυ, and u is the vector of additive effects of the remaining unmarked QTL with mean of zero and variance-covariance matrix Gu (i.e. animals effects, previously represented by a, are subdivided into υ and u, as a=KK+u, where K is the incidence matrix relating υ to a). Z are incidence matrices relating Kυ and u to y; e is a vector of residual effects with variance-covariance matrix R. To perform MA-BLUP, inverses of Gυ and Gu need to be calculated. The inverse Gu can be obtained as with Ga in regular BLUP (see above). The inverse for Gυ can be computed efficiently for large data sets where marker genotypes can be inferred on each animal and parental origin of marker is known (Fernando and Grossman, 1989), and in the case where marker genotypes are not known on some animal and parental origin of marker is unknown (Hoeschele, 1993; van Arendonk et al., 1994; Wang et al., 1991; Wang, et al., 1995).

As used herein the terms “marker” and “molecular genetic marker” (MME) preferably refer to a sequence of DNA that has a specific location on a chromosome that can be measured in a laboratory. To be useful, a marker needs to have two or more alleles or variants. Common types of markers include, but are not limited to: RFLP=restriction fragment length polymorphism; SSR=simple sequence repeat (a.k.a. “microsatellite” markers); and SNP=single nucleotide polymorphism. Markers can be either direct, that is, located within the gene or locus of interest, or indirect, that is closely linked with the gene or locus of interest (presumably due to a location which is proximate to, but not inside the gene or locus of interest). Moreover, markers can also include sequences which either do or do not modify the amino acid sequence of a gene.

As used herein the term “mixed model equation” preferably refers to a model for equations that solve for both random effects and fixed effects. The term random effects in the context of MA-BLUP is used to denote factors that have an unsystematic impact on the trait with levels that may represent a random distribution. Random effects will typically have levels that were not deliberately arranged by the experimenter (deliberately arranged factors may called fixed effects), but which were sampled from a population of possible samples instead. Linear models incorporating both fixed effects and random effects are called mixed linear models. The best linear unbiased prediction of random effects and fixed effects are the solution of the following linear equations, which are termed mixed model equations.

$$y = Xb + {Z}_{1} u + {Z}_{2} v + ⅇ \\ [ \begin{matrix}{X}^{′} {R}^{- 1} X & {X}^{′} {R}^{- 1} {Z}_{1} & {X}^{′} {R}^{- 1} {Z}_{2} \\ {Z}_{1}^{′} {R}^{- 1} X & {Z}_{1}^{′} {R}^{- 1} {Z}_{1} + {G}_{u}^{- 1} & {Z}_{1}^{′} {R}^{- 1} {Z}_{2} \\ {Z}_{2}^{′} {R}^{- 1} X & {Z}_{2}^{′} {R}^{- 1} {Z}_{1} & {Z}_{2}^{′} {R}^{- 1} {Z}_{2} + {G}_{v}^{- 1}\end{matrix} ] [ \begin{matrix}b \\ u \\ v\end{matrix} ] = [ \begin{matrix}{X}^{′} {R}^{- 1} y \\ {Z}_{1}^{′} {R}^{- 1} y \\ {Z}_{2}^{′} {R}^{- 1} y\end{matrix} ]$$

As used herein the preferred meaning for the term “marker assisted allocation” (MAA) is the use of phenotypic and genotypic information to identify animals with superior estimated breeding values (EBVs) and the further allocation of those animals to a specific use designed to optimize the improvement of the genetic merit of the animal population.

As used herein the term “meat quality trait” preferably means any of a group of traits that are related to the eating quality (or palatability) of pork. Examples of such traits include, but are not limited to muscle pH, purge loss (or water holding capacity), muscle color, firmness and marbling scores, intramuscular fat percentage, and tenderness.

As used herein the term “polymorphism” refers to the variation that exists in the DNA sequence for a specific marker or gene. That is, in order for a polymorphism to exist there must be more than one allele for a gene or marker.

As used herein the term “preconditioned conjugate gradient” preferably refers to a method for the symmetric positive definite linear system. The method proceeds by generating vector sequences of iterates that are successive approximations to the solution, with the residual corresponding to the iterates, and the search directions used in updating the iterates and residual.

As used herein the term “purge” (e.g. “loin purge”) preferably refers to the liquid escaping from the meat while in a vacuum sealed plastic package for a period of time (e.g. through the first 7-days, or through day 28).

As used herein a “qualitative trait” is one that has a small number of discrete categories of phenotypes and for which the genetic component is generally controlled by a small number of genes.

As used herein the term “quantitative trait” is used to denote a trait that is controlled by a large number of genes each of small to moderate effect. The observations on quantitative traits often follow a normal distribution.

As used herein the term “quantitative trait locus (QTL)” is used to describe a locus that contains polymorphism that has an effect on a quantitative trait.

As used herein the term “random genetic effects” is preferably used to denote factors with levels that were not deliberately arranged by the experimenter (those factors are called fixed effects), but that were, instead, sampled from a population of possible samples. A typical random genetic effect in animal breeding is additive genetic effect. Moreover, random genetic effects can be subdivided into at least two categories. “Continuous random genetic effects” that are “quantitative” effects that are governed by a plurality of genes, each of which contributes additively to the quality or trait. “Discontinuous random genetic effects” are categorical or qualitative and may be dependent on a single or few genetic loci.

As used herein the term “reproduction trait” refers to any of a group of traits that are related to animal reproduction, (e.g., swine reproduction and sow productivity). Examples in swine include, but are not limited to, number of piglets born per litter, piglet birth weight, piglet survival rate, pigs weaned per litter, litter weaning weight, age at puberty, farrowing rate, days to estrus, and semen quality.

As used herein the term “selection index” preferably refers to a weighted sum of EBVs for different economic traits. The selection index for each animal is a relative value and may be expressed in biological or economic units. Animals are ranked and selected based on the selection index. The values for the selection index are empirically and/or subjectively determined by analyzing the market values for a given trait. For example, suppose it is determined that a trait for “efficient growth” has tremendous future potential in the swine market and that two traits, 196-day body weight (bw) and lean percentage (lp) are used as metrics for efficient growth. Further suppose that through market analysis it is determined that each additional pound of 196-day bw is worth $0.40 and each additional lean percentage point is worth $2.00. In this model the selection weights for bw and lp are, respectively, $0.40 and $2.00. The Selection Index (I) is calculated according to the following equation:
I=(0.4)(EBV_{bw})+(2.0)(EVB_{Ip}).

Once the EBV is calculated, the selection index can be used as part of a herd management program or system to identify the specific animals most likely to produced offspring having the desired trait characteristics. It is noted that in order to be useful in a selection index the component EBVs must have all been simultaneously calculated, otherwise they would be of a different scale and not comparable.

Illustrative Embodiments

Various embodiments of the invention disclosed herein provides for marker-assisted best linear unbiased prediction (MA-BLUP) as part of methods and/or systems that provide a fully integrated genetic evaluation system. The MA-BLUP methods and systems disclosed herein combine traditional best linear unbiased prediction (BLUP) methodology with current marker-assisted selection (MAS) theory into a single yet robust computer executable algorithm useful to produce estimated breeding values (EBV) for each animal in a population. The theory and computing algorithms disclosed provide unexpectedly useful and effective extensions and modifications of previously known techniques.

Various embodiments of the present invention provide MA-BLUP implemented marker-assisted best linear unbiased prediction algorithms in a form that is functional and practical for use by breeding companies and/or large farming enterprises. The MA-BLUP methodology described herein provides for methods and/or systems that may be utilized to simultaneously analyze inputs of pedigree data, production performance data, and genetic marker data from a population and produce EBVs for each animal in the population as output.

Among the unique features of the MA-BLUP as herein disclosed is the ability to utilize molecular genetic information acquired from any method or form of genetic analysis including genotyping of candidate genes (i.e. genes of which certain variants are known or believed to provide economic other advantage when present). Other methods of genetic analysis are well known to those of ordinary skill in the art and include, but are not limited to, marker genotyping (which can be based on RFLPs=restriction fragment length polymorphisms; simple sequence repeat (SSR, a.k.a. “microsatellite” markers), polymerase chain reaction (PCR) amplified fragments, especially multiplexing PCR (the simultaneous amplification of several sequences in a single reaction)) and single nucleotide polymorphism (SNP, which analyzes single nucleotide differences in, for example, or near a gene of interest).

One particularly powerful aspect of the current invention is that it allows for the simultaneous analysis of three or more of these markers under multi-trait statistical models. Thus, the instant invention provides for methods and systems that allow those of skill in the art to evaluate an animal population with regards to pedigree information and a pre-selected list of one or more quantitative traits, one or more QTL for each quantitative trait, and three or more molecular genetic markers for each QTL. Moreover, the methods and systems provided allow the animals in the population to be ranked according to their EBV for a given trait or group of traits. Once the animals are ranked, this ranking information can then be used as part of a breeding management system to achieve the desired breeding goals. For example, it can be used to increase the population's average genetic merit for the selected trait(s) and/or it can be used to relatively quickly produce animals that have the genetic predisposition for highly favorable expression of a pre-selected trait.

Another powerful aspect of the instant invention that will be appreciated by those of skill in the art is that the MA-BLUP invention may be modified to provide for the analysis of any type of population through the use of a variety of “statistical models”. The various statistical models may be provided as input data in any of the embodiments of the instant invention.

Specifically statistical models are used to individually tailor the general MA-BLUP methodology to adapt to the specific data characteristics of the defined population. Thus, the instant invention provides for general purpose MA-BLUP analysis that is independent of the statistical models that any particular user may want to employ. For example, for molecular swine breeding one major statistical problem is determining estimated breeding values for each animal in a population using data that includes pedigree information, farm animal trait metrics (such as average daily weight gain, litter size, average weight at weaning, and etc.), and molecular genetic data. A statistical model for this problem would be:
y=Xb+Z₁u+Z₂v+e
where y is a vector of phenotypic data, b is a vector of fixed effects, u is a vector of polygenic effects and v is a vector of QTL (quantitative trait locus) effects. The variance-covariance matrices are Gᵤ for u and Gᵥ for v.

Moreover, as will be apparent to those skilled in the art statistical models for use with the instant invention will also require parameters such as the heritability of the selected traits and the genetic correlations between the selected traits. Also, the distance between markers and recombination rate between two markers are parameters also important to MA-BLUP

Another, aspect of various embodiments of the current invention is that the methods and systems disclosed allow for the effective “handling of missing terms”. That is not all data must be provided for each animal in a population. For example, the data may provide for pedigree data for some animals but not others. Similarly, phenotypic or genotypic (marker) data may be missing for some individual animals but not others. Thus, one powerful aspect of the instant invention is that it allows for the simultaneous analysis of various databases, including pedigree, phenotypic, and genotypic data that may have missing “terms” for any given animal.

Thus, through the use of different statistical models various embodiments of the instant invention are specifically tailored for methods, systems, and etc. for determining the EBV for a wide variety of organisms including, but not limited to, farm animals, such as swine, cattle, sheep, goats, poultry. Further, it is well within the ability of one of ordinary skill in the art provided with the instant disclosure, to design a statistical model for use in any desired population, plant or animal. In preferred aspects of these embodiments the population is made up of swine, cattle, or sheep. In a particularly preferred aspect of this embodiment the population is a swine population.

To aid in the speed and efficiency of the A-BLUP analysis various embodiments of the invention employ a pre-conditioned conjugate gradient (PCCG) algorithm with variable-size diagonal blocking as a pre-conditioner. When QTL effects are included in linear mixed model, we find it is more effective to take n by n block diagonal for polygenic portion and 2n by 2n block diagonal for QTL portion in linear equation systems as pre-conditioner, where n is the number of traits in the analysis. This pre-conditioning strategy is referred to as “variable-size block-diagonal pre-conditioning” algorithm. Comparing with diagonal pre-conditioning lgorithm which were previously used in common computer packages the variable-size block-diagonal pre-conditioning algorithm is 150% more effective in terms of computing time. This dramatically reduces computing time.

Pre-conditioning is a technique commonly used in linear algebra. For example, suppose one wants to solve the following linear equation: Ax=b.

A pre-conditioner is a matrix, “M”. The pre-conditioning process comprises multiplying the both side of the linear equation by M, that is MAx=Mb. It is noted that this pre-conditioning process has two features: it does not change solution and it makes solving process faster and solution more accurate (see Shewchuk, 1994).

Equation 1, below, provides the pseudocode of an algorithm to solve the problem Ca=r using the precondition conjugate gradient method, as provided in Stranden, I. and M. Lidauer, 1999, which is herein incorporated by reference.

$${a}^{( 0 )} ⟸ initial guess ;$$

$${r}_{0}^{( 0 )} ⟸ r - {Ca}^{( 0 )}$$

$${d}^{( 0 )} ⟸ {M}^{- 1} {r}_{0}^{( 0 )} ;$$

$${f}_{0} ⟸ {r}_{0}^{( 0 )} {d}^{( 0 )}$$

$$for$$

$$k = 1 , 2 , …$$

$${q}^{( k )} ⟸ {Cd}^{( k - 1 )} ;$$

$${α}_{k} ⟸ {f}_{k - 1} / {d}^{{( k )}^{′}} {q}^{( k )}$$

$${a}^{( k )} ⟸ {a}^{( k - 1 )} + {α}_{k} {d}^{( k - 1 )}$$

$$if k is divisible by 100$$

$${r}_{0}^{( k )} ⟸ r - {Ca}^{( k )}$$

$$else$$

$${r}_{0}^{( k )} ⟸ {r}_{0}^{( k - 1 )} - {α}_{k} {q}^{( k )}$$

$${s}^{( k )} ⟸ {M}^{- 1} {r}_{0}^{( k )}$$

$${f}_{k} ⟸ {r}_{0}^{{( k )}^{′}} {s}^{( k )}$$

$${β}_{k} ⟸ {f}_{k} / {f}_{k - 1}$$

$${d}^{( k )} ⟸ {s}^{( k )} + {β}_{k} {d}^{( k )}$$

if not convergent continue iteration end

The “M” employed by various aspects of the instant invention is a block-diagonal matrix. For the present example, assuming there are t traits. “M” consists of three parts:

$$y = Xb + {Z}_{1} u + {Z}_{2} v + ⅇ \\ [ \begin{matrix}{X}^{′} {R}^{- 1} X & {X}^{′} {R}^{- 1} {Z}_{1} & {X}^{′} {R}^{- 1} {Z}_{2} \\ {Z}_{1}^{′} {R}^{- 1} X & {Z}_{1}^{′} {R}^{- 1} {Z}_{1} + {G}_{u}^{- 1} & {Z}_{1}^{′} {R}^{- 1} {Z}_{2} \\ {Z}_{2}^{′} {R}^{- 1} X & {Z}_{2}^{′} {R}^{- 1} {Z}_{1} & {Z}_{2}^{′} {R}^{- 1} {Z}_{2} + {G}_{v}^{- 1}\end{matrix} ] [ \begin{matrix}b \\ u \\ v\end{matrix} ] = [ \begin{matrix}{X}^{′} {R}^{- 1} y \\ {Z}_{1}^{′} {R}^{- 1} y \\ {Z}_{2}^{′} {R}^{- 1} y\end{matrix} ]$$

(a) t by t blocks extracted from diagonals of the following (a block is a subset of the left hand side of the mixed model equation).:
X′R⁻¹X

(b) t by t blocks extracted from diagonals of the following
Z₁′R⁻¹Z₁+Gᵤ⁻¹

(c) 2t by 2t blocks extracted from diagonals of the following
Z₂′R⁻¹Z₂+Gᵥ⁻¹

Though previous BLUP programs implemented iteration-on-data (IOD) algorithms, these previous programs were only 50% as effective as that provided by the instant invention. This is due to the “pre-calculated and stored” algorithm implemented in the current invention. Steps that were time-consuming, but independent of the iteration-on-data steps (such as calculating individual contributing coefficients when computing the inverse of variance-covariance matrices for QTL) are pre-calculated and stored for later use in each iteration. An optimized order of matrix-vector multiplication is implemented in IOD.

Moreover, as disclosed herein, applicants have created methods and systems for applying and integrating variable-blocking algorithms and PCCG algorithms with iteration on data to provide surprisingly useful and powerful analysis of molecular genetic, character trait, and animal pedigree information that provides those involved in management of animal population with an effective means to ascertain and evaluate EBV for individual animals. These evaluations can then be utilized as part of a herd management system.

Additionally, various embodiments of the instant invention employ iteration-on-data methodology, which greatly reduces computer memory requirements.

Animals may be selected for use according to the instant invention by any suitable means; for example using computer programs or other means for recording parentage/pedigree and selecting the most suitable pairings. The use of computer programs can be further enhanced with the input of biometric data, including the use of molecular genetic analyses.

The methods and systems of the various embodiments of the instant invention employ computer algorithms for solving mixed model equations (MME) that take into account and provide output to guide breeding based on both fixed and random genetic effects (including both continuous random effects, such as additive genetic effects, and discontinuous or categorical random effects).

Various embodiments of the instant invention provide methods for improving an animal population's estimated breeding value or for identifying breeding pairs in order to quickly maximize the manifestation of a desirable trait. That is, the methods and systems of the present invention may be used to identify those potential parent animals that, when bred to one another, are most likely to manifest a maximum improvement of the selected trait in their progeny.

According to various aspects of this embodiment of the invention the methods comprise. (1) selecting one or more trait(s) for which population improvement is desired. (2) Providing for the animal population a database containing data on one or more quantitative traits loci. (3) Providing database(s) of data for the individual animals in the population where the database(s) comprise data for one, two, three, or more molecular genetic markers for each QTL for each trait for which improvement is desired. (4) Providing a database comprising the pedigree data for the animals in the population. (4) optionally providing data regarding fixed effects for the animals in the population. (5) (6) Providing and using a computer program capable of performing marker assisted best linear unbiased prediction to concurrently analyze the data from the databases provided and to calculate and provide, as an output of that calculation, an estimated breeding value (EBV) for each of the animals for the selected traits, and a ranking of the animals with respect to their individual estimated breeding values. A particular aspect of this embodiment of the invention provides for using the calculated EBVs to prepare a breeding plan for the animal population that provides for optimal improvement in the average genetic merit of the population or for maximizing the genetic merit of specific progeny.

In any aspect of the invention the number of traits selected and the number of quantitative trait loci (QTL) for each trait may be one or more. In a preferable aspect of the invention the number of QTLs selected for each trait may be 1, 2, 3, 4, 5, 6, 7, 8, 9, 10, 15, 20, or 30, or more. Moreover, in any aspect of the invention the number of molecular genetic markers for each QTL may be 1, 2, 3, 4, 5, 6, 7, 8, 9, 10, 11, 12, 13, 14, 15, 16, 17, 18, 19, 20, 25, or 30, or more. In preferred aspects of any embodiment of the invention the number of molecular genetic markers is 2 (two) or more. In even more preferred aspects of this embodiment the number of molecular genetic markers is three or more.

In preferred aspects of this embodiment of the invention, the markers linked to the QTL can form a marker haplotype. In this sense, a marker haplotype is a particular set of marker alleles from two or more neighboring markers that tend to be co-inherited. To be co-inherited, the markers making up the haplotype must be located relatively closely together (e.g. all markers would be located within a 5 cM interval). In even more preferred aspect of this embodiment, to increase the probability of co-inheritance, the markers forming the haplotype are located within an interval less than 1 cM wide. As an example, if 3 SNP markers were located closely enough to be co-inherited, and if theses markers had the following possible alleles,

| Markers: | Marker 1 | Marker 2 | Marker 3 |
|---|---|---|---|
| 1^{st} Allele | A | C | A |
| 2^{nd} Allele | T | G | C |

Then, the possible haplotypes would be as follows: ACA, ACC, AGA, AGC, TCA, TCC, TGA, TGC. These individual haplotypes can be inherited for several generations with little chance of recombination and, therefore, can be very important in terms of their linkage to the possible QTL alleles. As the number of alleles per marker or number of markers per haplotype increase, the number of possible haplotypes also increase, but in an exponential fashion. Therefore, the capability of the MA-BLUP methods and systems, described herein, to include several markers per QTL increases the informativeness of marker haplotypes linked to a QTL, thereby greatly increases the probability of finding linked markers as well as the probability of accurately tracking marked QTL alleles in successive generations. Moreover, the ability to use marker haplotypes increase the flexibility and robustness of the MA-BLUP program described herein.

In any aspects of this embodiment of the invention the type molecular genetic markers may be selected from, but not limited to, the group comprising: RFLPs (restriction fragment length polymorphisms), simple sequence repeat (SSR, a.k.a. “microsatellite” markers), polymerase chain reaction (PCR) amplified fragments, especially multiplexing PCR (the simultaneous amplification of several sequences in a single reaction) and single nucleotide polymorphisms (SNPs), which detect single nucleotide differences in, for example, a gene of interest). The markers information may also include data on point mutations, deletions, or translocations, or other gene isoforms. According to a particularly preferred aspect of this embodiment of the invention, the marker is selected from the group consisting of SNPs of the porcine PRKAG3 gene, variants in the porcine leptin receptor (pLEPR) gene, and the melanocortin-4-receptor (MC4R).

The melanocortin-4-receptor (MC4R) is described in three references each of which is herein incorporated by reference. These references include:
(1) Kim et al. Mammalian Genome (2002) 11(2): 131-5, which indicates that a missense variant of the porcine melanocortin-4 receptor (MC4R) gene is associated with fatness, growth, and feed intake traits.
(2) WO 00/06777 (Rothschild et al.; indicates that MC4R is marker for growth, feed intake and fat content). One polymorphism (a missense mutation Asp298His caused by a single nucleotide substitution G678A) in the MC4R gene was identified and found to be associated with growth rate, feed intake and fat content in swine. A RFLP based detection method is disclosed and used for genotyping. Additionally A TAQMAN®) based detection method is contemplated by the invention to detect the single nucleotide polymorphism.
(3) WO 01/075161 (Rothschild et al.; describes MC4R as marker for meat quality traits). The polymorphism (G678A) in MC4R gene is described as being associated with various meat quality traits including pH, drip loss, marble, and color in swine. A RFLP based detection method for genotyping is disclosed therein.

In any aspect of this embodiment of the invention the computer program may be configured to provide an evaluation of the “informativeness” and/or “closeness” of each molecular genetic marker with respect to the trait for which it serves as a marker. Accordingly, the methods and systems of the instant invention may be configured to determine which marker or markers are the most “informative” and which are the “closest” to the quantitative trait locus for which they serve as a marker.

The porcine leptin receptor (pLEPR) gene has been localized to chromosome 6, at approximately 122 centiMorgans (cM). Moreover, a number of DNA sequences (genomic and cDNA) for the porcine LEPR gene are available from the Genbank public DNA database, including: accession numbers: AF092422, AF167719, AF184173, AF184172, AH009271, AJ223163, AJ223162, U72070, AF036908, and U67739 (, each of which are herein incorporated by reference.

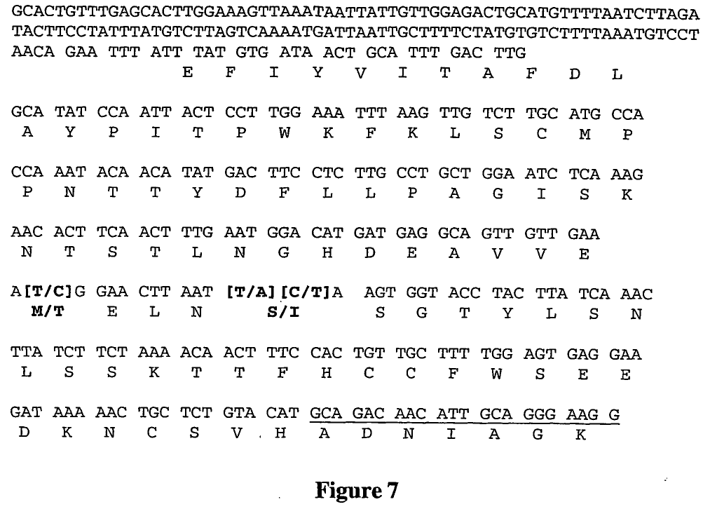

It has been shown that one useful allelic polymorphism comprises a “C/T” variation in the fourth exon of the leptin receptor gene. This variation results in the pLEPR protein produced from these variants having either a methionine or a threonine as amino acid number 69 of the prepro pLEPR protein (see FIG. 7). The C/T polymorphism results in either a cytosine (“C”) or thymine (“T”) variant at the nucleotide corresponding to position 609 of Genbank accession AF184172 in the fourth exon of the pLEPR gene. This polymorphism produces a pLEPR protein having either a methionine (if the nucleotide is “T”) or a threonine (if the nucleotide is “C”) at amino acid number 69 of the prepro pLEPR protein. The “T” variant (containing thymine, encoding methionine) is thought to be most common. As a shorthand designator, the polymorphism will be referred to as “the T69M” polymorphism.

An analysis of 2625 pigs from a single commercial line, showed that the presence of the “C” allele had a statistically significant correlation with a positive effect on: early ADG (average daily gain from day 0 to day 90 of life); late ADG (average daily gain from day 90 to day 165 of life), loin muscle pH, and loin muscle color, and drip loss. There was a small negative effect of the “C” allele on backfat, i.e. backfat was slightly increased.

In addition, ninety-seven (97) SNP markers, representing 38 loci on porcine chromosome 6 (SSC6) were genotyped on a panel of 1,444 pure line pigs from the a commercial line. The loci selected for SNP discovery were spread across an approximately 80 cM region on SSC6, which included the LEPR locus and the SNP producing the T69M mutation. Linkage disequilibrium analysis was used to identify both individual SNPs and SNP haplotypes (for up to three adjacent loci) that were significantly associated with growth-related phenotypes (i.e. backfat thickness, leanness, off-test weight and weight gain). All 97 SNPs and possible combinations of two and three adjacent SNP haplotypes were assessed for association with all phenotypes. Only four SNPs (plus several haplotypes containing these SNPs) were found to be significantly associated with backfat thickness, corrected for either age or weight. One of these SNPs included T69M and the other three mapped within 3 cM of T69M as estimated by linkage analysis.

Accordingly, instant invention may be employed using a marker for the pLEPR T69M mutant or any marker in linkage disequilibrium with such a marker.

In any embodiment of the instant invention the MA-BLUP program used may be integrated with a “scripting feature” that allows the user to manipulate the program algorithms using a scripting language that is similar to common English. For example if the program implementing MA-BLUP is written in the C++ computer programming language, the scripting feature allow the user to use the MA-BLUP program without knowing C++.

The instantly disclosed MA-BLUP provides methods and systems allowing those skilled in the art to analyze a collection of one, two, three or more markers for a given quantitative trait locus and determine the informativeness of the various markers. As noted in the definition's section, the “informativeness” of a given marker provides an indication as to how likely it is that an animal inheriting that marker will also express the desirable trait associated with that marker. Prior to the creation of MA-BLUP as used in the instantly disclosed invention, the best that could be said was that the presence of the marker indicated a 50:50 chance that the desirable trait would be present.

By providing a means for quantifying the informativeness of a given marker or set of markers, the instantly disclosed methods and systems provide a much better prognosticatory tool. The present invention provides methods and systems for determining which of a set of markers is the best predictor for a particular trait (i.e., is the most informative) and provides an indication of the proximity or closeness of the marker to the quantitative trait locus associated with a given trait.

Various embodiments of the instant invention provide for systems for increasing an animal populations average genetic merit for one or more pre-selected traits. The various invention embodiments also provide systems for rapidly improving a given trait in progeny by providing a means for selecting those animals from within the population that are most likely to effectively pass the germplasm for expressing the trait to their progeny. Systems according to this aspect of the invention comprise the following components. (1) A computer suitable for allowing the input of databases and/or execution of a program for calculating the EBVs of the animals using the methods described herein and providing for user access to and interface with the computer. (3) A computer accessible database or databases providing individual data for each animal in the population for each of one, two, three or more molecular genetic markers for a particular quantitative trait. (4) A computer accessible database providing individual pedigree data for each animal in the population. (5) Optionally, a computer accessible database providing individual data for each animal in the population for at least one trait of interest. (6) A computer executable program capable of using MA-BLUP to simultaneously evaluate the data in all databases and to rank the animals in the population according to their respective estimated breeding value. (7) A user interface, preferably including a data entry system, said user interface coupled to said computer and configured to allow the user to instruct the computer to access the available databases and use the MA-BLUP computer program to generate as output the EBV ranking of the animals and/or their individual estimated breeding values.

In preferred aspects of this embodiment of the invention, the animal population is selected from a swine herd, a bovine herd, and a ovine herd, although systems for evaluating any type of plant or animal population are envisioned as falling within the instant invention. In a particularly preferred embodiment the system is designed to evaluate swine herd estimated breeding values.

Those skilled in the art will appreciate that the methods and systems of the instant invention may be used to evaluate any type of molecular genetic marker. Accordingly, any specific markers described herein are meant to exemplary only and not to limit the scope of the invention in any way. Notwithstanding this fact, in particularly preferred embodiments of the invention the markers are selected from those that measure variation in the porcine PRKAG3 gene, porcine leptin receptor gene, and the MC4R gene.

In all embodiments of the invention the methods and systems may be used to evaluate an animal population's BV for a defined set of traits. Moreover, these methods and systems may be used to identify those individual animals or groups of animals that optimally provide the necessary germplasm to improve the frequency and/or quality of the desired trait. Meaning that the breeding pairs may be selected so as to optimize the expression of the selected trait in the progeny animals.

Other embodiments of the instant invention also provide for analysis and quantification of the relative predictive value of markers for quantitative trait loci. The invention provides for methods and systems that calculate the informativeness and/or closeness of a molecular genetic marker to the loci for the trait for which it serves as a marker. Moreover, with regard to quantitative trait markers, the methods and systems of the instant invention also provide an indication of the informativeness of the marker.

Various embodiments of the instant invention further provide for the use of the markers described supra. That is, the instant invention provides as one of its aspects, a means a means of using markers to identify those animals suitable for use in accordance with the invention. This process is termed MAS (marker assisted selection). The invention also envisions the use of MAA (marker assisted allocation). Through the use of MAA, selected animals are allocated for use so as to most effectively and efficiently bring about the desired genetic improvements in progeny animals.

In certain embodiments of the instant invention, information/data obtained from the analysis of various biometric measurements as well as other types of information (e.g., pedigree) can be weighted in a “selection index” in order to provide an evaluation of an animal's value as a parent, i.e., its estimated breeding value.

Phenotypic measures are affected (biased) by the herd and year or season in which the animal's performance is measured. In order to correct for this bias a procedure called BLUP (Best Linear Unbiased Prediction of breeding value) was developed (see, Animal Breeding, p. 84). As noted supra, there are currently several computer programs available from the authors of the software that can be used to calculate BLUP values.

Inbreeding is defined as the probability that two genes (i.e. alleles) at a locus are identical by descent (Malecot, 1948). The inbreeding level F_{X}) (i.e. inbreeding coefficient) can be calculated from pedigree records tracing back to the founder animals of a given population as follows:
F_{X}=(½)a_{XsXd}
(where, a_{XsXd} is the additive genetic relationship between Xs and Xd; if X is the progeny of Xs and Xd)

Increased homozygosity due to inbreeding is generally perceived to have deleterious side affects such as inbreeding depression (i.e. a decrease in performance in production, reproduction, and fitness traits) and decreased genetic variation leading to reduced rates of genetic gain over time.

Inbreeding rate, ΔF, is defined as the increase in the inbreeding coefficient in one generation (Falcaner and Mackay, 1996), and can be approximated by:
ΔF=⅛Nₘ+⅛N_{f}
Where, Nₘ and N_{f} are the numbers of males and females, respectively, contributing to the next generation.

As evident in this approximation, as fewer animals are selected as parents, inbreeding rate tends to increase. Unfortunately, increased selection pressure takes the form of selecting a smaller proportion of parents for the next generation. Therefore, swine breeding companies normally try to balance the extra genetic gain from selecting fewer parents against the resulting increase in inbreeding rate. Typically in swine populations, many females are selected to produce sufficient offspring for the next generation; therefore, inbreeding caused by female parents is not usually a concern. However, in order to limit the inbreeding rate and to maintain genetic variation in the herd it is common practice to select more males than are strictly needed for reproduction purposes. This practice limits both the rate of genetic progress in the GN and the speed at which changes can be made in gene frequency and trait direction. When several sires must be selected as parents, it is difficult to find a set of sires that all have high breeding values with a particular genetic profile (e.g. specific genetic marker profile).

Limitations Due to Multi-Trait Selection Indexes:

Typically, selection in a population is practiced via the use of a multi-trait selection index. In this approach, estimated breeding values are calculated for each economic trait for each animal based on pedigree and phenotypic information. The estimated breeding values are then weighted according to the relative economic value of each trait as well as the intended direction of selection for the population and incorporated into a single, multi-trait selection index. These multi-trait indexes incorporate several sources of information for each animal (e.g. phenotypic records on ancestors, progeny and the animal itself). Selection indexes determine the long-term genetic progress for the population and must be carefully constructed to balance needs of both the present and future marketplaces. Accordingly, if temporary changes in the market occur, a breeding company cannot justify completely changing the selection index to reflect those changes; especially if future market conditions are not likely to match the current, temporary conditions.

Two-Stage Selection

Typically, selection takes place on quantitative traits based on BLUP breeding values and ranked in a multiple-trait selection index. However, there are increasing numbers of economic trait loci (ETL) that have been discovered that have been reported to be associated with traits that are not normally considered in the multiple-trait selection index yet have a measurable economic value (e.g. health or meat quality traits).

A simple approach to use of these genes is through two-stage selection. In the first stage, animals could be genotyped for one or more ETL then pre-selected for the most favorable form (allele) of the ETL. Next, in the second stage, additional selection is performed on the remaining animals according to the traditional multi-trait selection index. This approach has the benefit of being relatively easy to apply and may reduce the number of animals for which regular phenotyping is necessary (e.g. gain on test, ultrasound measures of back fat and loin eye area, etc.).

Alternatively, the first stage can comprise a standard phenotyping procedures and rankings according to multi-trait MA-BLUP EBVs. This is then followed by a second stage in which animals are differentiated according to their genotypes at one or more ETL. This second option does not present any savings in phenotyping, but could provide savings in genotyping if some animals rank too lowly to be considered for selection and therefore genotyping costs are not justified. In addition, some genotypes may have more value to certain customers than others and, therefore, marker-assisted allocation (MAA) can be used to allocate specify animals to customers desiring a particular genotype. MAA can therefore be justified by charging a premium to customers receiving the specified genotype.

Single-Stage (Multi-trait Index) Selection

Simultaneously incorporating all available information at the time of selection, in the form of a single-stage multi-trait selection index, is the most efficient form of selection. Moreover this method results in the greatest long-term progress towards the stated breeding objective. Other selection strategies such as two-stage selection (above), tandem selection (i.e. alternating selection on different traits over multiple generations), or use of independent culling levels (i.e. eliminate animals not reaching a minimum culling threshold) have been shown to be less efficient than index selection (Van Vleck, et al., 1987). Nevertheless, these other methods are sometimes employed for reasons related to ease of use, cost or speed of implementation.

Index selection normally takes the form of a linear equation, as follows:
Hᵢ=υ₁A₁ᵢ+υ₂A₂ᵢ+ . . . +υ_{N}A_{Ni}
where, Hᵢ is the selection index value for animal i, vᵢ, v₂ and v_{N} are the net economic values per unit of trait 1 through N, A₁ᵢ, A₂ᵢ and A_{Ni} are the additive genetic value for animal i for traits 1 through N. Additive genetic values for each trait can be calculated to include ETL information via MA-BLUP (described above). Further information is easily available regarding index selection (Van Vleck et al., 1987; Van Vleck, 1983).

One of the most difficult aspects of incorporating ETL information into multi-trait index selection is determining how to properly weight the new information relative to traditional trait phenotypic information. Since ETL information is often conditional on marker genotype information, this information can be difficult to include, because markers are not usually located directly at the ETL, but rather some distance from it. Recombination (chromosomal crossovers) can break down the linkage (strength of association) between the marker and the ETL, and tends to occur in proportion to the distance between the marker and the actual ETL. This recombination rate needs to be taken into account as well as situations where genotypes are not available on all animals.

This process has become much more feasible with the advent of MA-BLUP methodology (see above), whereby the ETL information is combined into the additive genetic breeding value for that trait for the animal. In the MA-BLUP scenario, marker information can be simultaneously included with phenotypic and pedigree information to predict breeding values. If the trait affected by the ETL is already included in the multi-trait selection index, then ranking and selection can proceed more or less as previously described.

However, if the ETL affects a new trait that is not currently in the breeding objective, then additional work must be done. First, to assess the economic value of the new trait and, second, to estimate the necessary genetic parameters surrounding the new trait (i.e. heritability, genetic variance and covariance with the other traits in the selection objective). Information regarding estimating genetic parameters and applications for BLUP models used in animal breeding is known to those of skill in the art (see, e.g. Henderson, 1984).

PRKAG3

The PRKAG3 gene encodes the gamma subunit of the porcine AMPK (adenosine monophosphate-activated protein kinase), which enzyme has been shown to play a key role in the regulation of energy metabolism in eukaryotic cells (Milan et al 2000). Animals having certain variants of the PRKAG3 gene have been shown to possess more desirable characteristics with regard to loin and ham pH, to have reduced seven-day purge from loin muscle, to have reduced drip loss, and other meat quality traits.

In accordance with various embodiments of the current invention MA-BLUP may be used to rank the EBV of animals in a pig population based, inter alia, on the animal's complement of various PRKAG3 SNPs. That is, based on the animals' haplotype for the PRKAG3 gene. According to the various aspects of this embodiment of the invention the EBV rankings of the herd population are then used as part of a herd management/breeding program useful to improve the average genetic merit for meat quality traits in general and specifically with respect to the meat quality traits influenced by the animal's PRKAG3 haplotype.

Various embodiment of the invention provide for methods, kits, and compositions that are drawn to the use of SNPs from the porcine PRKAG3 gene. Aspects of this embodiment of the invention are useful for enhancing one or more meat quality traits. The enhanced meat quality traits include all those commonly measured by those skilled in the art. In preferred aspects of this embodiment of the invention the meat quality traits are selected from the group consisting of increased loin pH, increased ham pH, reduced 7-day purge and reduced drip loss.

Certain aspects of this embodiment of the invention provide methods for enhancing the meat quality traits of animals in a herd and/or for the screening of a plurality of animals in a herd to identify the nature of the PRKAG3 haplotypes present in the screened animals. Next those pigs identified as having one or more desired allele are used as part of a breeding plan to produce offspring having a increased frequency of the desired allele and/or trait. In a preferred aspect of this embodiments the SNPs are selected from one or more of the known SNPs in the porcine PRKAG3 gene. In a more preferred embodiment of the invention the SNPs are selected from the group consisting of: an A/G at position 51, A/G at position 462, A/G at position 1011, C/T at position 1053, C/T at position 2475, A/G at position 2607, A/G at position 2906, A/G at position 2994, and C/T at position 4506 (note that the numbering provided above is according to the sequence of SEQ ID NO:1). It is noted that the selecting process may include the use of the MA-BLUP program described herein.

Any suitable method for screening the animals for their status with respect to the newly described PRKAG3 polymorphisms is considered to be part of the instant invention. Such methods include, but are not limited to: DNA sequencing, restriction fragment length polymorphism (RFLP) analysis, heteroduplex analysis, single stand conformational polymorphism (SSCP) analysis, denaturing gradient gel electrophoresis (DGGE), real time PCR analysis (TAQMAN®), temperature gradient gel electrophoresis (TGGE), primer extension, allele-specific hybridization, and INVADER® genetic analysis assays.

### EXAMPLES

The following examples are examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

### Example 1

### MC4R Maker Marker Used in a Commercial Pig Line A

From approximately 600 young animals out of a performance testing station the top 10 of males were selected for incorporation into breeding herd to produce the next generation of animals.

Phenotypic Data

| animal | sex | litter | cgp | age | wda | leanp |
|---|---|---|---|---|---|---|
| 0000001016391 | M | 20047 | 90006 | 160 | 109 | — |
| 0000001030745 | M | 20048 | 90006 | 164 | — | 552 |
| 0000005010960 | M | 20049 | 90172 | 170 | 169 | 500 |
| 0000005010985 | M | 20050 | 90172 | 174 | 141 | 536 |
| 0000005010986 | M | 20050 | 90172 | 167 | 141 | 515 |
| 0000005010987 | M | 20050 | 90172 | 174 | 118 | 545 |
| 0000005011018 | F | 20050 | 90172 | 167 | 113 | 601 |
| 0000005011019 | F | 20050 | 90172 | 167 | 113 | 515 |
| 0000005011020 | F | 20050 | 90172 | 167 | 119 | 552 |
| 0000005011021 | F | 20050 | 90172 | 167 | 106 | 546 |
| . | | | | | | |
| . | | | | | | |
| . | | | | | | |
| 2220000007490 | M | 34789 | 90682 | 154 | 103 | 492 |
| 2220000007494 | M | 34789 | 90682 | 154 | 127 | 511 |
| 2220000007497 | F | 34789 | 90682 | 154 | 115 | 533 |
| 2220000007498 | F | 34789 | 90682 | 154 | 96 | 520 |
| 2220000007499 | M | 34790 | 90682 | 154 | 131 | 525 |
| 2220000007501 | M | 34790 | 90682 | 154 | 140 | 534 |
| 2220000007503 | F | 34790 | 90682 | 154 | 136 | 511 |
| 2220000007505 | F | 34790 | 90682 | 154 | 110 | 508 |
| 2220000006486 | F | 34796 | 90682 | 152 | 124 | 531 |
| 2220000006487 | F | 34796 | 90682 | 152 | 80 | 556 |

Genotypic Data

| animal | genotype |
|---|---|
| 0009705450992 | A/G |
| 0009705451278 | A/G |
| 0009705451281 | A/G |
| 0009705451282 | A/G |
| 0009705451288 | A/G |
| 0009705456787 | G/G |
| 0009709501525 | A/G |
| 0009709501528 | A/G |
| 0009709501530 | G/G |
| 0009709501531 | G/G |
| . | |
| . | |
| . | |
| 2220000006032 | A/G |
| 2220000006033 | A/G |
| 2220000006034 | G/G |
| 2220000006035 | A/G |
| 2220000006036 | A/G |
| 2220000006037 | G/G |
| 2220000006038 | G/G |
| 2220000006039 | G/G |
| 2220000006040 | A/G |
| 2220000006041 | G/G |

Pedigree Data

| animal | sire | dam | sex |
|---|---|---|---|
| 0000009000347 | 0000009000345 | 0000009000346 | M |
| 0000009000245 | 0000009000351 | 0000009000352 | M |
| 0000009000367 | 0000009000361 | 0000009000366 | M |
| 0000009000350 | 0000009000348 | 0000009000349 | M |
| 0000009000363 | 0000009000361 | 0000009000362 | M |
| 0000009000365 | 0000009000269 | 0000009000364 | M |
| 0000009000358 | 0000009000347 | 0000009000357 | M |
| 0000009000344 | 0000009000221 | 0000009000276 | M |
| 0000009000360 | 0000009000227 | 0000009000359 | M |
| 0000009000334 | 0000009000269 | 0000009000333 | M |
| . | | | |
| . | | | |
| . | | | |
| 2220000008593 | 1090000024220 | 1090000021806 | F |
| 2220000008594 | 1090000024220 | 1090000021806 | F |
| 2220000008595 | 1090000024220 | 1090000021806 | F |
| 2220000008596 | 1090000024220 | 1090000021806 | F |
| 2220000006876 | 1130000051724 | 1090000024984 | M |
| 2220000006877 | 1130000051724 | 1090000024984 | M |
| 2220000006878 | 1130000051724 | 1090000024984 | M |
| 2220000006879 | 1130000051724 | 1090000024984 | F |
| 2220000006880 | 1130000051724 | 1090000024984 | F |
| 2220000007516 | 1130000051724 | 1100000031328 | F |

Statistical Model
There are Two Traits: Weights Per Day of Age (wda) and Lean Percentage (Leanp).
wda=age age*age sex cgp mc4r litter animal

leanp=age age*age sex cgp mc4r litter animal

Animal Ranking

Rank of animals

| | | | not using | using |
|---|---|---|---|---|
| pigId | sex | MC4R | marker | marker |
| 1130000063582 | M | A/G | 1 | 1 |
| 1130000062299 | M | A/G | 2 | 2 |
| 1130000062304 | M | A/G | 4 | 3 |
| 1130000063592 | M | A/G | 5 | 4 |
| 1050000027328 | M | A/G | 6 | 5 |
| 1130000063593 | M | A/G | 7 | 6 |
| 1130000063501 | M | A/A | 19 | 7 |
| 1130000061796 | M | A/A | 20 | 8 |
| 1090000025391 | M | G/G | 3 | 9 |
| 1130000063574 | M | A/A | 22 | 10 |

### Example 2

### Identification of New SNPs in the PRKAG3 Gene and their Use for Improving EBV for Meat Quality Traits in Swine Herds

The porcine PRKAG3 gene is expressed exclusively in skeletal muscle and is involved in the regulation of glycogen synthesis. There is now convincing evidence in the art that supports the hypothesis that mutations in this gene affect meat quality traits such as glycolytic potential (GP, is an indicator of the glycogen level in a living animal which is calculated as a total of the total principle compound susceptible to conversion to lactate. GP equals 2 (glycogen+glucose+glucose-6-phosphate)+lactate), pH, drip loss, and purge. At least two different single nucleotide polymorphisms (SNPs) that alter the amino acid sequence of the mature protein have been found in exons for this gene. Moreover, these polymorphisms have been shown to be associated with the meat quality traits listed above.

For example, there are two separate international patent applications (WO 01/20003 A2 and WO 02/20850 A2) drawn to the use of these SNPs. Disclosed herein are nine (9) newly identified PRKAG3 SNPs that have been shown to be associated with meat quality traits.

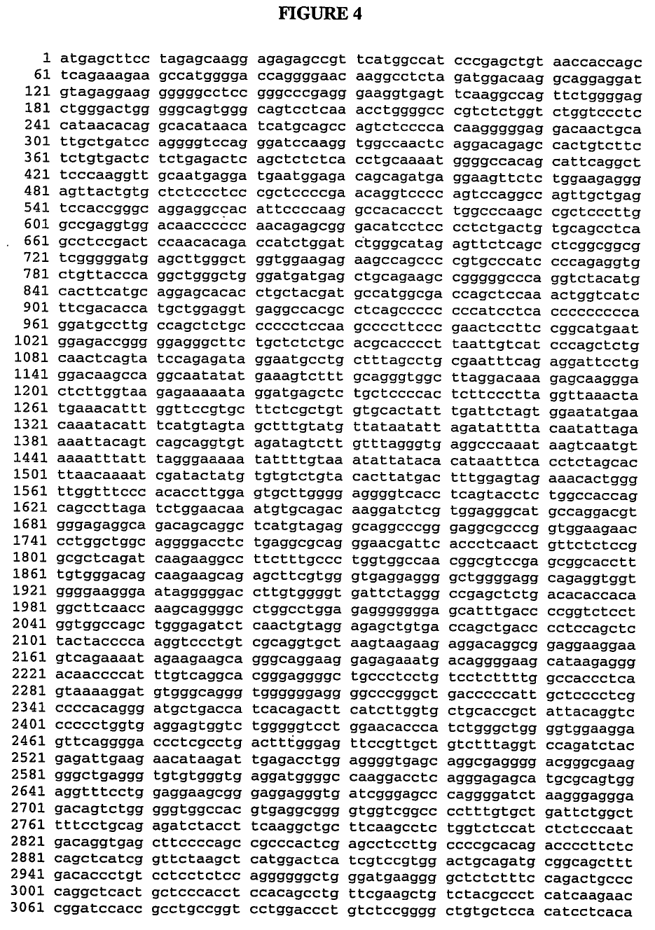

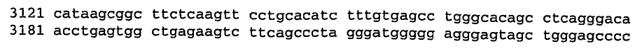

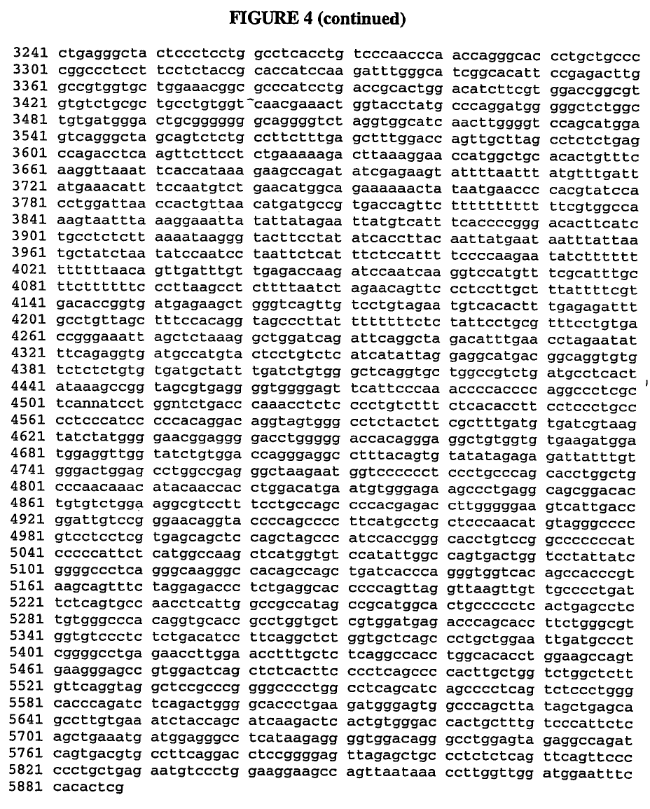

The sequence of the porcine AMPK (AMP-activated protein kinase) available as Genbank Accession number AF214521 (see FIG. 4), was used to prepare primers for use to amplify fragments representing the majority of the known sequence for this gene (see Table 1 for the primer pair sequences)

TABLE 1
Primer names and sequences used to amplify PRKAG3 for SNP discovery

| Amplicon | Forward | Forward | Reverse | Reverse | Amplicon |
|---|---|---|---|---|---|
| Name | Primer Name | Primer Sequence | Primer Name | Primer Sequence | size (bp) |
| RN7-636 | RN7-636-F | TTCCTAGAGCAAGG | RN7-636-R | GATGTCCCGCTCTG | 629 |
| | | AGAGAGC | | TTGG | |
| RN826- | RN826- | GCCCAGGTCTACAT | RN826- | ATTTGGGCCTCACC | 604 |
| 1430 | 1430F | GCACTT | 1430R | CTAAAC | |
| RN1611- | RN-F1613 | GCCACCAGCAGCCT | PRKAG3-R | CCCTTCCCCACCAC | 318 |
| 1929 | | TAGAT | | CTCT | |
| RN2170- | RN2170- | TAGAAGAAGCAGGG | RN2170- | GCAGGAAAAGCCAG | 598 |
| 2768 | 2768F | CAGGAA | 2768R | AATCAG | |
| RN2807- | RN2807- | CCATCTCTCCCAAT | RN2807- | GGTCCACGAAGATG | 608 |
| 3415 | 3415F | GACAGG | 3415R | TCCAGT | |
| RN3558- | RN3558- | CTGCCTTCTTTGAG | RN3558- | TCACCGGTGTCACG | 593 |
| 4151 | 4151F | CTTTGG | 4151R | AAAATA | |
| RN4242- | RN4242- | ATTCCTGCGTTTCC | RN4242- | TTCTCCCACATTCA | 599 |
| 4841 | 4841F | TGTGAC | 4841R | TGTCCA | |
| RN5056- | RN5656- | CCAAGCTCATGGTG | RN5056- | TTCACAAGGCTGCT | 594 |
| 5650 | 5650F | TCCATA | 5650R | CAGCTA | |

Genomic DNA from twelve (12) unrelated animals from a commercial pig line “A” was used as template for amplifications using the eight primer pairs, set out in Table 1 as primers. Following amplification, the resulting amplicons were sequenced and the sequences from all 12 animals were aligned, amplicon by amplicon, and evaluated to identify potential sequence polymorphisms. Twenty-four (24) SNPs were identified, including several of the SNPs identified in the (WO 01/20003 A2 and WO 02/20850 A2) patent applications. TAQMAN® SNP assays were designed and validated for 11 of these SNPs, including nine SNPs that were previously unknown (see Table 2).

TABLE 2
PRKAG3 SNPS FOR WHICH TAQMAN ® assays were successfully validated

| | | | | | Nucleotide | | |
|---|---|---|---|---|---|---|---|
| Amplicon | Sequence | SNP | | SNP | position in | Amino acid | Discovered |
| Name | ID | Assay # | SNP Name | Alleles | AF214521 | change | by |
| RN7-636 | 1464167 | 156331 | 231_22 | AG | 51 | NO | Monsanto |
| RN7-636 | 1464167 | 156330 | 231_60 | AC | 89 | YES | Milan et al. |
| | | | | | | (N30T) | |
| RN7-636 | 1459459 | 148001 | 231-433 | AG | 462 | NO | Monsanto |
| RN826-1430 | 1459460 | 148002 | 230_613 | AG | 1011 | NO | Monsanto |
| RN826-1430 | 1459460 | 148003 | 230_571 | CT | 1053 | NO | Monsanto |
| RN1611-1929 | 1459461 | 148004 | 221_57 | CT | 1845 | YES | Milan et al. |
| | | | | | | (V199I) | |
| RN2170-2768 | 1459462 | 148006 | 228_320 | CT | 2475 | NO | Monsanto |
| RN2170-2768 | 1459462 | 148008 | 228_452 | AG | 2607 | NO | Monsanto |
| RN2807-3415 | 1459463 | 148009 | 227_77 | AG | 2906 | NO | Monsanto |
| RN2807-3415 | 1459463 | 148010 | 227_165 | AG | 2994 | NO | Monsanto |
| RN4242-4841 | 1459464 | 148012 | 225_245 | CT | 4509 | NO | Monsanto |

These SNPs were next genotyped on a panel of 2,693 animals from two different commercial lines, “A′” and “B”, representing 118 half-sib families with meat quality phenotypes. SNP haplotypes were determined for as many of the animals as possible and association analysis was carried out to determine which haplotypes were most predictive/informative for the various meat quality traits.

Although there are theoretically 2¹¹ different haplotype groups possible with 11 different SNPs, nearly 95% of the animals for which haplotypes could be completely determined had one of only three different haplotypes (see Table 3). One particular haplotype (Hap. Group 2) was significantly (p<0.001) associated with increased pH in both loin and ham. Further, this Hap. Group 2 was also associated with reduced 7-day purge from loin muscle (see Tables 4 and 5).

TABLE 3
Major SNP haplotypes for the eleven PRKAG3 SNPs genotyped
on the A′ commercial pig line population panel

| | SNP | Hap. | Hap. | Hap. | |
|---|---|---|---|---|---|
| SNP | Assay # | Group 1 | Group 2 | Group 3 | Others |
| g51a | 156331 | G | G | A | |
| g89t | 156330 | G | G | T | |
| g462a | 148001 | G | G | A | |
| t1011c | 148002 | T | T | C | |
| g1053a | 148003 | G | G | A | |
| g1845a | 148004 | G | A | G | |
| c2475t | 148006 | C | C | T | |
| t2607c | 148008 | T | T | C | |
| g2906a | 148009 | G | G | A | |
| g2994a | 148010 | G | G | A | |
| a4509g | 148012 | A | A | G | |
| | Frequency | 0.377 | 0.269 | 0.302 | 0.052 |

TABLE 4
Average allele effect estimate for haplotype Groups 1, 2 & 3.

| Trait | Hap. Group 1 | Hap. Group 2 | Hap. Group 3 |
|---|---|---|---|
| 7 day purge | 0.0124 | −0.0889 | 0.0637 |
| Ham pH | 0.0022 | 0.0261 | −0.0260 |
| Loin pH | 0.0032 | 0.0142 | −0.0167 |

TABLE 5
Impact of haplotype fixation

| Trait | Hap. Group 1 | Hap. Group 2 | Hap. Group 3 |
|---|---|---|---|
| 7 day purge | 0.0103 | −0.01339 | 0.1571 |
| Ham pH | 0.0074 | 0.0772 | −0.0289 |
| Loin pH | 0.0097 | 0.0298 | −0.0279 |

As can be seen from Table 3, which shows the three major haplotype groups, all of the SNPs, with the exception of c1845t (SNP assay 148004) were in almost complete linkage disequilibrium with each other. Thus, a genotype for any one of the 10 SNPs (besides c1845t) we genotyped in PRKAG3 is predictive, with a high degree of confidence, of the genotype at any of the other nine SNPs.

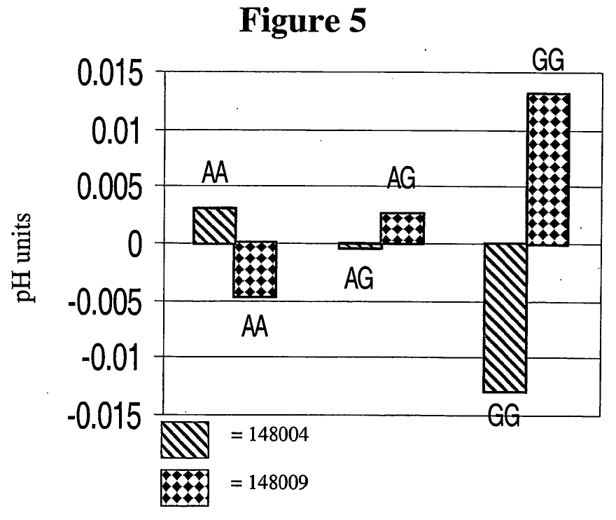

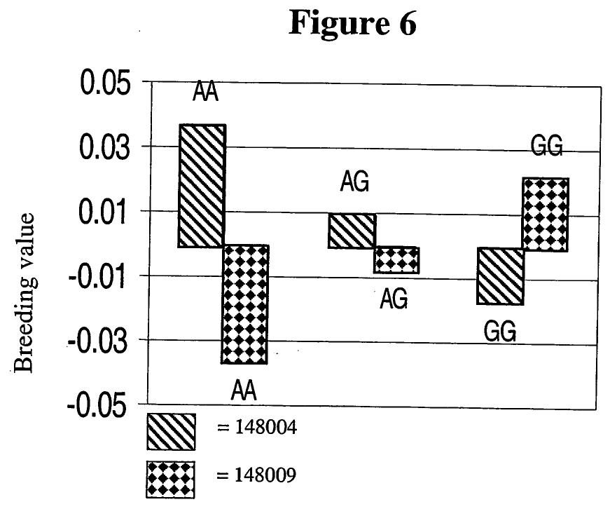

FIGS. 5 and 6 show the genotype and breeding values, respectively, for SNP c1845t (SNP assay #148004) and SNP a2906g (SNP assay #148009), which is representative of the ten SNPs in almost completed linkage disequilibrium. The favorable allele of 148004 for increased pH and decreased 7-day purge is the “A” allele, whereas the favorable allele for these traits for 148009 is the “G” allele. As is demonstrated by these figures (and also by Table 6) 148004 accounts for a greater degree of variation in meat pH than 148009 (i.e. it is either a causal mutation or is in greater linkage disequilibrium with the causal mutation). However, selection for the G allele of 148009 (or the favorable alleles of the other nine markers found to be in linkage disequilibrium with 148009) can also be used to select animals in commercial line A for improved meat quality traits of pH and 7-day purge.

TABLE 6
Gene effects and breeding values for SNPs 148004 (004) and 148009 (009)

| PRKAG3 gene effects: | AA | AG | GG |
|---|---|---|---|
| GV | a | d | −a |

| marker | Genotype Counts | | | Sum |
|---|---|---|---|---|
| 004 | 333 | 1185 | 1335 | 2853 |
| 009 | 468 | 1290 | 1287 | 3045 |

| | | | | | | check |
|---|---|---|---|---|---|---|
| Marker | freq A (p) | freq G (g) | freq AA | freq AG | freq GG | (sum freq) |
| 004 | 0.324395373 | 0.675604627 | 0.116719243 | 0.415352261 | 0.467928496 | 1 |
| 009 | 0.365517241 | 0.634482759 | 0.153694581 | 0.42364532 | 0.422660099 | 1 |

| | | | | | | | Gene Subst |
|---|---|---|---|---|---|---|---|
| Marker | a | d | GV AA | GV AG | GV GG | Sum | (alpha) |
| 004 | 0.0274 | −0.0012 | 0.003198107 | −0.000498423 | −0.012821241 | −0.010121556 | 0.026978549 |
| 009 | −0.0308 | 0.0062 | −0.004733793 | 0.002626601 | 0.013017931 | 0.010910739 | −0.029132414 |

| | Mid-Homo | Pop | | | | check | Impact of | Impact of |
|---|---|---|---|---|---|---|---|---|
| marker | Mean | Mean | BV AA | BV AG | BV GG | (mean BV) | Fixing A | Fixing G |
| 004 | 5.890121556 | 5.88 | 0.036453665 | 0.009475116 | −0.017503433 | 0 | 0.036453665 | −0.017503433 |
| 009 | 5.869089261 | 5.88 | −0.036968029 | −0.007835615 | 0.021296799 | 0 | −0.036968029 | 0.021296799 |

Genotypic Values

| Marker | AA | AG | GG |
|---|---|---|---|
| 004 | 0.003198107 | −0.000498423 | −0.012821241 |
| 009 | −0.004733793 | 0.002626601 | 0.013017931 |

Breeding Values

| marker | AA | AG | GG |
|---|---|---|---|
| 004 | 0.036453665 | 0.009475116 | −0.017503433 |
| 009 | −0.036968029 | −0.007835615 | 0.021296799 |

| Haplotype Freq.: | | |
|---|---|---|
| 004/009 Haplotype | Count | Freq. |
| A/A | 1 | 0.000468165 |
| A/G | 679 | 0.317883895 |
| G/A | 918 | 0.429775281 |
| G/G | 538 | 0.251872659 |
| Total | 2136 | 1 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the methods described herein without departing from the concept the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention as defined by the appended claims.

### Example 3

### PRKAG3 Marker Used in a Commercial Pig line A′

Analysis was done on 60 boars coming out of the performance testing station in March, 2003. The top 10 of them were selected for introduction into the breeding herd to produce next generation. Two SNP markers were used in MA-BLUP for the following calculations.

Phenotypic Data

| animal | dam | sex | gline | litter | cgp | cgp3 | age | wda | leanp | pH |
|---|---|---|---|---|---|---|---|---|---|---|
| 0000000628060 | 0000000103005 | F | 16 | 21597 | 90442 | 0 | 152 | 139 | 501 | — |
| 0000000499339 | 0000000452451 | F | 15 | 21600 | 90442 | 0 | 151 | 154 | 502 | — |
| 0000000499340 | 0000000452451 | F | 15 | 21600 | 90442 | 0 | 151 | 132 | 511 | — |
| 0000000499341 | 0000000452386 | F | 15 | 21601 | 90442 | 0 | 151 | 149 | 463 | — |
| 0000000499342 | 0000000452386 | F | 15 | 21601 | 90442 | 0 | 151 | 129 | 454 | — |
| 0000000499343 | 0000000452270 | F | 15 | 21602 | 90442 | 0 | 151 | 137 | 510 | — |
| 0000000499314 | 0000000452747 | F | 15 | 21603 | 90442 | 0 | 150 | 147 | 472 | — |
| 0000000499315 | 0000000452747 | F | 15 | 21603 | 90442 | 0 | 150 | 133 | 487 | — |
| 0000000499316 | 0000000452010 | F | 15 | 21604 | 90442 | 0 | 150 | 145 | 456 | — |
| 0000000499317 | 0000000452010 | F | 15 | 21604 | 90442 | 0 | 150 | 143 | 502 | — |
| . | | | | | | | | | | |
| . | | | | | | | | | | |
| . | | | | | | | | | | |
| 1070000010847 | 1130000056726 | F | 16 | 32809 | 90422 | 699 | 172 | 140 | 501 | 610 |
| 1070000010875 | 1130000054850 | F | 16 | 32810 | 90422 | 699 | 172 | 145 | 528 | 634 |
| 1070000010877 | 1130000054850 | F | 16 | 32810 | 90422 | 699 | 171 | 148 | — | 602 |
| 1070000010899 | 1130000056380 | F | 16 | 32811 | 90422 | 699 | 171 | 143 | 499 | 604 |
| 1070000010901 | 1130000056380 | F | 16 | 32811 | 90422 | 0 | 171 | 137 | 485 | — |
| 1070000010903 | 1130000056380 | F | 16 | 32811 | 90422 | 699 | 171 | 143 | 496 | 607 |
| 2220000002623 | 1090000025314 | F | 15 | 32813 | 90505 | 0 | 178 | 112 | 543 | — |
| 2220000002624 | 1090000025314 | F | 15 | 32813 | 90505 | 0 | 178 | 116 | 552 | — |
| 2220000002625 | 1090000025314 | F | 15 | 32813 | 90505 | 0 | 178 | 83 | — | — |
| 2220000002626 | 1090000025314 | F | 15 | 32813 | 90505 | 0 | 178 | 112 | 544 | — |

Genotypic Data

| animal | m004 | m009 |
|---|---|---|
| 0001995120096 | G/G | G/G |
| 0001996264361 | G/G | A/G |
| 0001996229682 | G/G | G/G |
| 0001996237608 | G/G | A/G |
| 0009645400235 | A/G | G/G |
| 0009645408986 | G/G | A/G |
| 0009652443262 | G/G | G/G |
| 0009652443205 | . | G/G |
| 0009652450481 | G/G | A/G |
| 0009652424155 | G/G | A/G |
| . | | |
| . | | |
| . | | |
| 2220000005567 | A/G | A/G |
| 2220000005568 | A/G | G/G |
| 2220000005569 | A/G | G/G |
| 2220000005570 | G/G | A/G |
| 2220000005571 | G/G | A/G |
| 2220000005572 | G/G | A/A |
| 2220000004935 | G/G | G/G |
| 2220000004936 | G/G | G/G |
| 2220000004937 | A/G | G/G |
| 2220000004938 | A/G | G/G |

Pedigree Data

| animal | sire | dam | sex |
|---|---|---|---|
| 0000000449871 | 0000000449568 | 0000000449554 | M |
| 0000000449875 | 0000000449568 | 0000000449554 | F |
| 0000000449876 | 0000000449568 | 0000000449554 | F |
| 0000000449878 | 0000000449568 | 0000000449554 | F |
| 0000000449870 | 0000000449565 | 0000000449562 | M |
| 0000000449877 | 0000000449565 | 0000000449562 | F |
| 0000000449881 | 0000000449565 | 0000000449562 | F |
| 0000000449872 | 0000000449564 | 0000000449563 | M |
| 0000000449879 | 0000000449564 | 0000000449563 | F |
| 0000000449882 | 0000000449564 | 0000000449563 | F |
| . | | | |
| . | | | |
| . | | | |
| 2220000006808 | 1090000024991 | 1130000054009 | F |
| 2220000006809 | 1090000024991 | 1090000024710 | M |
| 2220000006810 | 1090000024991 | 1090000024710 | M |
| 2220000006811 | 1090000024991 | 1090000024710 | M |
| 2220000006812 | 1090000024991 | 1090000024710 | M |
| 2220000006813 | 1090000024991 | 1090000024710 | M |
| 2220000006814 | 1090000024991 | 1090000024710 | F |
| 2220000006815 | 1090000024991 | 1090000024710 | F |
| 2220000006816 | 1090000024991 | 1090000024710 | F |
| 2220000006817 | 1090000024991 | 1090000024710 | F |

Statistical Model
wda=age sex gline cgp litter animal
leanp=age sex gline cgp litter animal

pH=gline m004 cgp3 dam animal

Animal Ranking

Rank of animals

| | | | not using | using |
|---|---|---|---|---|
| pigId | sex | PRKAG3 | marker | marker |
| 1130000060709 | M | | 3 | 1 |
| 1060000011461 | F | | 2 | 2 |
| 1130000060712 | M | | 8 | 3 |
| 1060000011463 | M | GG | 1 | 4 |
| 1130000060715 | M | | 11 | 5 |
| 1130000060716 | M | | 13 | 6 |
| 1070000007452 | M | | 4 | 7 |
| 1060000011362 | F | | 6 | 8 |
| 1130000061484 | F | AG | 67 | 9 |
| 1130000060710 | M | | 25 | 10 |

SSR Makers used in a research line: 79 boars came out of the performance testing station in March, 2003. Top 10 of them were selected into the breeding herd to produce next generation. 26 QTLs and 55 SSR markers used in MA-BLUP to select the top 10 boars.

Pedigree Data

| animal | sire | dam | sex |
|---|---|---|---|
| 0000000449554 | 0 | 0 | . |
| 0000000449558 | 0 | 0 | . |
| 0000000449562 | 0 | 0 | . |
| 0000000449563 | 0 | 0 | . |
| 0000000449564 | 0 | 0 | . |
| 0000000449565 | 0 | 0 | . |
| 0000000449566 | 0 | 0 | . |
| 0000000449568 | 0 | 0 | . |
| 0000000449573 | 0 | 0 | . |
| 0000000449579 | 0 | 0 | . |
| . | | | |
| . | | | |
| . | | | |
| 1130000062981 | 1020000011792 | 1020000012539 | F |
| 1130000062982 | 1020000011792 | 1020000012539 | F |
| 1130000062983 | 1020000011792 | 1020000012539 | F |
| 1130000062984 | 1020000011792 | 1020000012539 | F |
| 1130000062941 | 1020000011715 | 1020000011830 | M |
| 1130000062942 | 1020000011715 | 1020000011830 | M |
| 1130000062943 | 1020000011715 | 1020000011830 | M |
| 1130000062944 | 1020000011715 | 1020000011830 | M |
| 1130000062945 | 1020000011715 | 1020000011830 | M |
| 1130000062946 | 1020000011715 | 1020000011830 | M |

$$Statistical Model$$

$$bf = sex cg 196 age 196 litt mc 4 r\_a mc4r\_d bf\_q 1 bf\_q 5 bf\_q6 bf\_q12 bf\_q16 animal$$

$$lea = sex cg 196 age 196 litt mc 4 r\_a mc4r\_d lea\_q 2 lea\_q 3 lea\_q7 lea\_q8 lea\_q12 animal$$

$$wt = sex cg 196 age 196 litt mc 4 r\_a mc4r\_d wt\_q 1 wt\_q2 wt\_q4 wt\_q5 wt\_q6 wt\_q7 wt\_q8 wt\_q9 wt\_q10 animal$$

$$dfi = sex batch wt 90 litt mc4r\_a mc4r\_d dfi\_q1 dfi\_q 6 dfi\_q8 dfi\_qF11 dfi\_q12 animal$$

Animal Ranking

Rank of animals

| | | not using | using |
|---|---|---|---|
| pigId | sex | marker | marker |
| 1130000059813 | M | 2 | 1 |
| 1130000060009 | M | 1 | 2 |
| 1130000059458 | M | 5 | 3 |
| 1130000060506 | M | 6 | 4 |
| 1130000059571 | M | 4 | 5 |
| 1130000059449 | M | 8 | 6 |
| 1130000060523 | M | 3 | 7 |
| 1130000059471 | M | 7 | 8 |
| 1130000059607 | M | 9 | 9 |
| 1130000059676 | M | 11 | 10 |

### Example 4

### Conjugate Gradient Algorithms

Given the inputs A,b, a starting value x, a (perhaps implicitly defined) preconditioner M, a maximum number of iterations iₘₐₓ and error tolerance [epsilon]<1:

$$\vert \begin{matrix}i ⟸ 0 \\ r ⟸ b - Ax \\ d ⟸ {M}^{- 1} r \\ {δ}_{nex} ⟸ {r}^{T} d \\ {δ}_{0} ⟸ {δ}_{new} \\ While i < {i}_{max} and {δ}_{new} > {[ epsilon ]}^{2} {δ}_{0} do \\ q ⟸ Ad \\ α ⟸ \frac{{δ}_{new}}{{d}^{T} q} \\ x ⟸ x + α d \\ r ⟸ r - α q \\ s ⟸ {M}^{- 1} r \\ {δ}_{old} ⟸ {δ}_{new} \\ {δ}_{new} ⟸ {r}^{T} s \\ β ⟸ \frac{{δ}_{new}}{{δ}_{old}} \\ d ⟸ s + β d \\ i ⟸ i + 1 \\ End\end{matrix} \vert$$

### Example 5

### Accommodation to Multiple Markers (Determining Informativeness)

Consider a chromosome fragment containing a quantitative trait locus(QTL) and one set of markers (N₁,N₂, . . . ,Nₙ) on the left side of QTL and another set of markers (M₁,M₂, . . . ,Mₘ) on the right side of QTL.
Nₙ . . . N₂N₁Q M₁M₂ . . . Mₘ

The instant invention provides algorithms to detect a set of informative flanking markers (Nᵢ,Mⱼ) near QTL. This algorithm works like a resizable window moving around the chromosome fragment to locate a set of informative flanking markers, one is on the left side of QTL and another on the right side of QTL. The following example illustrates that N₁ and M₂ is a set of markers that is closest to QTL and informative (linkage phase is known).

$$\frac{\begin{matrix} & & & & & & & & & & \\ & & & & & & & & & & \end{matrix}}{\begin{matrix} & & & & & & & & \\ & & & & & & & & \end{matrix}} \vert \frac{\begin{matrix} & & & \\ {N}_{1} & Q & & {M}_{2}\end{matrix}}{\begin{matrix} & & & \end{matrix}} \vert \frac{\begin{matrix} & & & & & & & \\ & & & & & & & \end{matrix}}{\begin{matrix} & & & & & \\ & & & & & \end{matrix}}$$

### Example 6

### Variable-Size Block-Diagonal Pre-Conditioning

Solving the mixed model equations using pre-conditioning conjugate gradient (PCCG) is the core part of MA-BLUP. The equations can be expressed in the matrix notation assuming there are 6 animals involved:

$$[ \begin{matrix}{a}_{11} {a}_{12} {a}_{13} {a}_{14} {a}_{15} {a}_{16} \\ {a}_{21} {a}_{22} {a}_{23} {a}_{24} {a}_{25} {a}_{26} \\ {a}_{31} {a}_{32} {a}_{33} {a}_{34} {a}_{35} {a}_{36} \\ {a}_{41} {a}_{42} {a}_{43} {a}_{44} {a}_{45} {a}_{46} \\ {a}_{51} {a}_{52} {a}_{53} {a}_{54} {a}_{55} {a}_{56} \\ {a}_{61} {a}_{62} {a}_{63} {a}_{64} {a}_{65} {a}_{66}\end{matrix} ] [ \begin{matrix}{x}_{1} \\ {x}_{2} \\ {x}_{3} \\ {x}_{4} \\ {x}_{5} \\ {x}_{6}\end{matrix} ] = [ \begin{matrix}{b}_{1} \\ {b}_{2} \\ {b}_{3} \\ {b}_{4} \\ {b}_{5} \\ {b}_{6}\end{matrix} ] \qquad ( 1 )$$

The diagonal elements (a₁₁, a₂₂, . . . ,a₆₆) are most commonly used for pre-conditioning. Constant-size block-diagonal such as

$$[ \begin{matrix}{a}_{11} {a}_{12} \\ {a}_{21} {a}_{22}\end{matrix} ] , \\ [ \begin{matrix}{a}_{33} {a}_{34} \\ {a}_{43} {a}_{44}\end{matrix} ] , \\ [ \begin{matrix}{a}_{55} {a}_{56} \\ {a}_{65} {a}_{66}\end{matrix} ]$$

are recommended in the literature for pre-conditioning. In contrast, the methods and systems of the instant invention provide for the use of variable-size block-diagonal such as

$$[ {a}_{11} ] , \\ [ \begin{matrix}{a}_{22} {a}_{23} \\ {a}_{32} {a}_{33}\end{matrix} ] , \\ [ \begin{matrix}{a}_{44} {a}_{45} {a}_{46} \\ {a}_{54} {a}_{55} {a}_{56} \\ {a}_{64} {a}_{65} {a}_{66}\end{matrix} ]$$

The size of each block-diagonal is determined by the nature of MA-BLUP mixed model equations.

Iteration On Data (IOD) Combined with PCCG

Due to the nature of mixed model equations, the most elements in equation(1), above are zeros. MA-BLUP first processes data and stores the non-zeros contributed from each record of data to the mixed model equation in the hard disk. MA-BLUP does not actually build up elements, aᵢⱼ's, in the computer memory. It only stores xᵢ's, bᵢ's and block-diagonals. Accordingly, the methods and systems of the instant invention provide for algorithms that iterate over each data record again and again till it converges.

### Example 7

### Comparison of Analysis According to the Instant Invention with Previously Existing Program, ISU-MABLUP

The Iowa State University (ISU) program is based on the public version of Matvec. Testing was carried out comparing the speed and efficiency of a MA-BLUP according to the instant invention with the ISU package. The comparisons for speed are shown in the unit of either minute(m), hour(h), or day(d) when it is appropriate.

7.1 Using ISU Data Sets

ISU-MABLUP comes with its own testing data sets, which will be used to compare two packages.

7.1.1 Small Data Sets

These are simulated data with 14 animals. The number of traits and QTL for each QTL model are shown below.

TABLE 7

| | 1 QTL | 2 QTLs |
|---|---|---|
| 1-trait | model 1 | model 2 |
| 2-trait | model 3 | model 4 |

Both the ISU package and presently disclosed invention generate the ‘identical’ (indicated by ‘+’) results for each of the above four QTL models. The meaning of ‘identical’ results has two folds (1) it refers only as to estimable function value (2) it refers only as to the first four digits after the decimal-point.

TABLE 8

| | Linux | | Computer Farm | |
|---|---|---|---|---|
| | Direct solver | IOD solver | Direct solver | IOD solver |
| ISU-MABLUP | + | + | + | + |
| Present | + | + | + | + |
| invention | | | | |

7.1.2 Large Data Sets

There are two traits, two QTLs and 12,643 animals. Both ISU package and presently disclosed invention generate the ‘identical’ results.

Using Larger Data Sets

Two data sets of approximately 63,000 animals were used. One data set contains one QTL and another contains two QTLs. An extensive test and comparison of the IOD solver was done since it is one of the most robust and efficient solvers available in MABLUP analysis. Two platforms were used. They are 32-bit Intel PC with Linux and a cluster of 64-bit Sparcstation with Solaris (Computer Farm). All tests generated ‘identical’ results. The speed, however, were varied from platform to platform, from single trait to multiple trait. The comparisons for speed are shown in next three tables.

7.2.0.1 One QTL

TABLE 9

| | |
|---|---|
| Linux | Computer Farm |

| | 3-trait | 4-trait | 3-trait | 4-trait |
|---|---|---|---|---|
| ISU-MABLUP | 5 h | 7 h | 15 h | 29 h |
| Present Invention | 2 h | 3.5 h | 11 h | 17 h |

7.2.0.2 Two QTL

TABLE 10

| | |
|---|---|
| Linux | Computer Farm |

| | 3-trait | 4-trait | 3-trait | 4-trait |
|---|---|---|---|---|
| ISU-MABLUP | 11 h | 16 h | 41 h | 63 h |
| Present Invention | 4 h | 8 h | 24 h | 25 h |

7.2.0.3 No QTL

In order to examine any differences of polygenic effect resulted from incorporation of QTL associated with marker in the genetic evaluation system, we re-run MABLUP without QTL in the linear model. The data set used is one containing one QTL.

TABLE 11

| | |
|---|---|
| Linux | Computer Farm |

| | 3-trait | 4-trait | 3-trait | 4-trait |
|---|---|---|---|---|
| ISU-MABLUP | 43 m | 108 m | 190 m | 449 m |
| Present Invention | 7 m | 25 m | 41 m | 136 m |

7.3 Present Invention Versus MTDFREML

Using a different data set comprising four traits and 28,624 animals. The comparison for speed is given below in the unit of minute(m). Note that we used the fastest solver (IOC_PCCG) in the aspect of the present invention used.

TABLE 12

| | Linux | Charlie |
|---|---|---|
| MTDFREML | 6 m | — |
| Present invention | 3 m | 9 m |

### Example 8

### Computing the Inbreeding Coefficient for a QTL

The conditional probability that two homologous alleles at the marker linked QTL (MQTL) in individual loci i are identical by descent, gives Gobs is defined as the inbreeding coefficient for a QTL;
fᵢ=Pr(Qᵢ¹≡Qᵢ²|G_{obs})

This is different from Wright's inbreeding coefficient, which is the conditional probability that two homologous alleles at any locus in individual i are identical by descent, given only the pedigree.

The pair of two homologous alleles at the MQTL, Qᵢ¹ and Qᵢ², in individual i descended from one of the following parental pairs:
(Qₛ¹,Q_{d}¹),(Qₛ¹,Q_{d}²),(Qₛ²,Q_{d}¹) or (Qₛ²,Q_{d}²)
Let Tₖ_{ₛ}ₖ_{_{d}} denote the event that the pair of alleles in i descended from the parental pair (Qₛ^{k}^{ₛ}, Q_{d}^{k}^{_{d}}) for kₛ,k_{d}=1 or 2. Now, if fᵢ can be written as:

$${f}_{i} = {\sum }_{{k}_{s} = 1}^{2} {\sum }_{{k}_{d} = 1}^{2} Pr ( {Q}_{s}^{{k}_{s}} ≡ {Q}_{d}^{{k}_{d}} \text{\textbar} {G}_{obs} ) Pr ( {T}_{{k}_{s} {k}_{d}} \text{\textbar} {G}_{obs} )$$

Then Pr (Tₖ_{ₛ}ₖ_{_{d}}|G_{obs}) can be expressed in terms of the probability of descent for a QTL allele as, for example:

$$Pr ( {T}_{11} \text{\textbar} {G}_{obs} ) = \frac{{B}_{i} ( 1 , 1 ) {B}_{i} ( 2 , 3 )}{{B}_{i} ( 1 , 1 ) + {B}_{i} ( 1 , 2 )} + \frac{{B}_{i} ( 1 , 3 ) {B}_{i} ( 2 , 1 )}{{B}_{i} ( 1 , 3 ) + {B}_{i} ( 1 , 4 )}$$

where Bᵢ(l,k) are the probability of descent for QTL allele k to allele l.

### REFERENCES

The following references, to the extent that they provide exemplary procedural or other details supplementary to those set forth herein, are specifically incorporated herein by reference.
Abdel-Azim G. and A. E. Freeman. 2001. A rapid method for computing the inverse of the gametic covariance matrix between relatives for a marked quantitative trait locus. Genet. Sel. Evol., 33:153-173.
Chakraborty, R., Moreau, L., Dekkers, J. C. 2002. A method to optimize selection on multiple identified quantitative trait loci. Genet. Sel. Evol. 34(2): 145-70.
Falconer, D. S. and Mackay, Introduction to Quantitative Genetics, T. F. C., Eds., Longman Group Limited, Longman House, Burnt Mill, Harlow Essex 2JE, England. 4^{th} Edition, 1986.
Fernando, R. L. and Grossman, M. 1989. “Marker assisted selection using best linear unbiased prediction,” Genet. Sel. Evol. 21:467-477.
Gibson, J. P. 1994. Short-term gain at the expense of long-term response with selection of identified loci. Proceedings of the 5^{th} World Congress on Genetics Applied to Livestock Production, Guelph, 21:201-204.
Henderson, C. R. 1984. Applications of Linear Models in Animal Breeding. Published by the University of Guelph, Guelph, Ontario, Canada.
Hernandez-Sanchez, J., Visscher, P., Plastow, G. and Haley, C. 2003. Candidate Gene Analysis for Quantitative Traits Using the Transmission Disequilibrium Test: The Example of the Melanocortin 4-Receptor in Pigs. Genetics. 164:637-644.
Kim, K. S., Larsen, N., Short, T., Plastow, G. and Rothschild, M. F. 2000. A missense variant of the porcine melanocortin-4-receptor (MC4R) gene is associated with fatness, growth, and feed intake traits. Mammalian Genome. 11:131-135.
Lidauer, M., Strandén, I., Mäntysaari, E. A., Pösö, J., and A. Kettunen. 1999, “Solving large test-day models by iteration on data and preconditioned conjugate gradient,” J. Dairy Sci. 82:2788-2796.
Malécot, G., 1948 Les Mathematiques de l'Heredite. Masson, Paris.)
Milan, D., et al. 2000. “A mutation in PRKAG3 associated with excess glycogen content in pig skeletal muscle. Science, 288:1248-1251.
Pong-Wong, R., George, A. W., Woolliams, J. A., and C. S. Haley. 2001. “A simple and rapid method for calculating identity-by-descent matrices using multiple markers,” Genet. Sel. Evol. 33:453-471.
Quaas, R. L., Anderson, R. D., Gilmour, A. R., 1984. BLUP school handbook; Use of mixed models for prediction and estimation of (co)variance components. Animal Breeding and Genetics Unit, University of New England, N.S.W. 2351, Australia.
Strandén, I. and M. Lidauer. 1999. “Solving large mixed linear models using preconditioned conjugate gradient iteration,” J. Dairy Sci. 88:2779-2787.
Shewchuk, J. R. 1994 “An introduction to the conjugate gradient method without the agonizing pain. Tech. Rep. CMU-CS-94-125, Carnegie Mellon University, Pittsburgh, Pa.
Totir, L. R. 2002. Genetic evaluation with finite locus models. PhD Dissertation. Iowa State University, Ames, Iowa.
Tsuruta, S., Misztal, I., and I. Strandén. 2001. “Use of the preconditioned conjugate gradient algorithm as a generic solver for mixed-model equations in animal breeding applications,” J. Animal Sci. 79:1166-1172.
Van Vleck, L. D., Pollak, E. J., and Oltenacu, E. A. B., Genetics for the Animal Sciences, W. H. Freeman and Company, New York, 1987
Wang, T., Fernando, R. L., van der Beek, S., Grossman, M., and J. A. M. van Arendonk. 1995. “Covariance between relatives for a marked quantitative trait locus.” Genet. Sel. Evol. 27:251-274
Wang, T., Fernando, R. L., Stricker, C. and R. C. Elston. 1996 “An approximation to the likelihood for a pedigree with loops.” Theor. Appl. Genet. 93:1299-1309.
WO 02/20850 A2, Rothschild et al., Mar. 14, 2002.

1. A method of increasing an animal population's average genetic merit, comprising;
a. selecting one or more traits for which an improved genetic merit is desired:
b. selecting one or more quantitative trait locus (QTL) for each selected trait;
c. selecting three or more molecular genetic markers of interest for each QTL for each selected trait;
d. providing databases comprising:
i. genotype data for three or more molecular genetic markers for each selected trait, for a plurality of animals in the population;
ii. data providing the pedigree for each animal in the population;
iii. optionally, data for one or more fixed effects;
e. using a computer program capable of performing a marker assisted best linear unbiased prediction to simultaneously analyze the data from the provided databases to calculate a ranking of the animals; wherein the computer program uses a variable-size block-diagonal preconditioned gradient (PCCG) algorithm to rank the animals; wherein the animals are ranked according to their estimated breeding value (EBV) for the selected molecular genetic markers and, if provided, quantitative traits.

2. The method of 1 further comprising using the calculated EBVs to prepare a breeding plan for the animal population that provides for optimal improvement in the genetic merit of the population.

3. The method of claim 1 wherein the animal population is a swine herd.

4. The method of claim 1 wherein the trait is selected from the group consisting of: efficient growth traits, meat quality traits, reproduction traits, and health traits.

5. The method of claim 1 wherein the molecular genetic markers are selected from any polymorphism known to affect expression of the mRNA or protein from a gene.

6. The method of claim 5 where the polymorphism is selected from the group consisting of: single nucleotide polymorphisms, simple sequence repeats, protein point mutations, and gene isoforms.

7. The method of claim 3 wherein at least one molecular genetic marker is selected from those markers known to modulate a favorable phenotype.

8. The method of claim 3 wherein at least one of the molecular genetic markers is a marker for selected from the group consisting of: a single nucleotide polymorphism in the porcine PRKAG3 (protein kinase, AMP-activated gamma-3 subunit) gene, and a polymorphism in the porcine melanocortin-4-receptor.

9. The method of claim 3 wherein at least one of the molecular genetic markers is a marker for a single nucleotide polymorphism in the porcine PRKAG3 gene.

10. The method of claim 1 wherein the computer program uses an iteration-on-data (IOD) algorithm.

11. (canceled)

12. The method of claim 1 wherein the output of the computer program further comprises results that indicate the informativeness of one or more of the selected molecular genetic marker for at least one quantitative trait locus (QTL) and/or a calculation of the genetic closeness/proximity of one or more molecular markers to at least one QTL.

13. The method of claim 12 wherein the molecular genetic markers having the highest degree of informativeness and/or closeness for at least one QTL are identified.

14. The method of claim 1 wherein the computer program utilizes a scripting feature to improve the ease of user interface.

15. The method of claim 1 wherein the selected molecular genetic markers comprise a marker haplotype.

16. A system for increasing an animal population's average genetic merit for at one or more selected traits, the system comprising:
a. a computer;
b. a computer accessible database providing data on one or more quantitative trait locus (QTL) for each selected trait;
c. a computer accessible database providing data, for animals in population, for three or more molecular genetic markers for each selected QTL for each selected trait;
d. a computer accessible database providing pedigree data for animals in the population;
e. optionally, a computer accessible database providing individual data for each animal in the population for at least one fixed effect;
f. a computer program capable of performing marker-assisted best linear unbiased prediction and simultaneously evaluating the data in all databases and ranking the animals in the population according to their respective estimated breeding value for each of the selected traits; wherein the computer program uses a variable-size block-diagonal preconditioned gradient (PCCG) algorithm to rank the animals;
g. a user interface including a data entry system, said user interface coupled to said computer and configured to allow the user to instruct the computer to access the available databases and use the computer program to generate output that includes a ranking of the animals according to their estimated breeding values and/or their individual estimated breeding values.

17. The system of claim 16 wherein the animal population is a swine herd.

18. The system of claim 17 wherein at least one of the molecular genetic markers is selected from the group consisting of markers for the porcine PRKAG3 gene and the gene encoding the melanocortin-4-receptor.

19. The system of claim 17 wherein at least one of the molecular genetic markers is a marker for a single nucleotide polymorphism in the porcine PRKAG3 gene.

20. The system of claim 17 wherein the selected molecular genetic markers comprise a marker haplotype.

21. A system for identifying the molecular genetic marker(s) having the highest degree of informativeness for one or more selected quantitative trait locus (QTL), the system comprising:
a. a computer;
b. a computer accessible database providing individual data, for animals in population, for three or more molecular genetic markers for each selected quantitative trait locus;
c. a computer program capable of simultaneously evaluating the data in all databases and determining the relative informativeness for each of the molecular genetic markers for which data is provided; wherein the computer program is capable of performing marker-assisted best linear unbiased prediction and uses a variable-size block-diagonal preconditioned gradient (PCCG) algorithm to determine the relative informativeness of each molecular genetic marker;
d. a user interface including a data entry system, said user interface coupled to said computer and configured to allow the user to instruct the computer to access the available databases and use the computer program to generate output that includes a indication of the informativeness of each molecular genetic marker for which data was provided.

22. The system of claim 21 wherein the quantitative trait locus is selected from any locus known to be associated with a known trait.

23. The system of claim 21 wherein the quantitative trait locus is selected from any locus for traits selected from the group consisting of efficient growth traits, meat quality traits, reproduction traits, and health traits.

24. The system of claim 21 further comprising providing computer accessible database(s) containing individual data for animals in the population for at least one fixed effect;
wherein the computer executable program is capable of simultaneously evaluating the data in all provided databases and ranking the animals in the population according to their respective estimated breeding value for each of the selected traits.

25. The system of claim 21 wherein the selected molecular genetic markers comprise a marker haplotype.

26-28. (canceled)

29. The method of claim 1 further comprising using the animals' ranks to identify the optimal breeding pairs in the population.

30. The method of claim 29 wherein the selected molecular genetic markers comprise a marker haplotype.

31. A method of enhancing one or more meat quality trait(s) in pigs, the method comprising:
a) screening a plurality of pigs to identify the nature of one or more single nucleotide polymorphisms (SNPs) in the porcine PRKAG3 gene, wherein said SNP(s) is/are selected from the group consisting of: an A/G at position 51, A/G at position 462, A/G at position 1011, C/T at position 1053, C/T at position 2475, A/G at position 2607, A/G at position 2906, A/G at position 2994, and C/T at position 4506, wherein all numbering is according to the sequence of SEQ ID NO:1 and identifying those having a desired allele;
b) selecting those pigs identified as having a desired allele;
c) using the selected pigs as sires/dams in a breeding plan to produce offspring; wherein the offspring have an increase frequency of the desired allele.

32. The method of claim 31 wherein the presence or absence of the polymorphism is determined by a method selected from the group consisting of: DNA sequencing, restriction fragment length polymorphism (RFLP) analysis, heteroduplex analysis, single strand conformational polymorphism (SSCP) analysis, denaturing gradient gel electrophoresis (DGGE), real time PCR analysis (TAQMAN®), temperature gradient gel electrophoresis (TGGE), primer extension, allele-specific hybridization, and INVADER® genetic analysis assays.

33. The method of claim 31 wherein at least one meat quality trait is selected from the group consisting of increased pH and decreased 7-day purge.

34. A kit for detecting the nature of one or more polymorphisms in the porcine PRKAG3) gene; the kit comprising a means for detecting for detecting the polymorphism in the DNA and or RNA from the gene;
wherein the polymorphisms are selected from the group consisting of one or more of the following SNP(s): an A/G at position 51, A/G at position 462, A/G at position 1011, C/T at position 1053, C/T at position 2475, A/G at position 2607, A/G at position 2906, A/G at position 2994, and C/T at position 4506, wherein all numbering is according to the sequence of SEQ ID NO:1.

35. The kit of claim 34 whereby the polymorphism is detected by one or more of the following means of detection: DNA sequencing, restriction fragment length polymorphism (RFLP) analysis, heteroduplex analysis, single strand conformational polymorphism (SSCP), denaturing gradient gel electrophoresis (DGGE), polymerase chain reaction (PCR), real time PCR analysis (TAQMAN®), temperature gradient gel electrophoresis (TGGE), enzyme linked immunosorbent assay (ELISA) and other immunoassay;
wherein the kit comprises one or more of the following: a restriction endonuclease enzyme, a DNA polymerase, a reverse transcriptase, a buffer, deoxyribonucleotides, an oligonucleotide suitable for use as a DNA or RNA probe, an oligonucleotide suitable for use as a primer in DNA or RNA synthesis, a fluorescent marker, and an antibody.

36. An oligonucleotide suitable for use in a kit according to claim 35.

37. The oligonucleotide of claim 36 selected from primers comprising the sequence of any of the primers listed in Table 1 (SEQ ID NO:2-17).

38. The oligonucleotide of claim 36 selected from the group consisting of the primers provided in Table 1 (SEQ ID NO:2-17).

39-46. (canceled)
